SEQUENCE LISTING (60 sequences)

SEQ ID NO: 1 - protein, 72 aa, Finegoldia magna
SEEEVTIKANLIFANGSTQTAEFKGTFEKATSEAYAYADTLKKDNGEYTVDVADKGYTLNIKFAGKEKTPEE

SEQ ID NO: 2 - protein, 72 aa, Finegoldia magna
PKEEVTIKANLIYADGKTQTAEFKGTFEEAAAEAYRYADALKKDNGEYTVDVADKGYTLNIKFAGKEKTPEE

SEQ ID NO: 3 - protein, 72 aa, Finegoldia magna
PKEEVTIKANLIYADGKTQTAEFKGTFEEATAEAYRYADLLAKENGKYTVDVADKGYTLNIKFAGKEKTPEE

SEQ ID NO: 4 - protein, 74 aa, Finegoldia magna
PKEEVTIKANLIYADGKTQTAEFKGTFAEATAEAYRYADLLAKENGKYTADLEDGGYTINIRFAGKKVDEKPEE

SEQ ID NO: 5 - protein, 74 aa, Finegoldia magna
PKEEVTIKVNLIFADGKTQTAEFKGTFEEATAKAYAYADLLAKENGEYTADLEDGGNTINIKFAGKETPETPEE

SEQ ID NO: 6 - protein, 74 aa, Finegoldia magna
PKEEVTIKVNLIFADGKIQTAEFKGTFEEATAKAYAYANLLAKENGEYTADLEDGGNTINIKFAGKETPETPEE

SEQ ID NO: 7 - protein, 74 aa, Finegoldia magna
PKEEVTIKVNLIFADGKTQTAEFKGTFEEATAEAYRYADLLAKVNGEYTADLEDGGYTINIKFAGKEQPGENPG

SEQ ID NO: 8 - protein, 72 aa, synthetic construct
PKEEVTIKANLIFADGKTQTAEFKGTFEEATAEAYRYADLLAKVNGEYTADLEDGGYTINIKFAGKEQPGEN

SEQ ID NO: 9 - protein, 70 aa, Finegoldia magna
KEQVTIKENIYFEDGTVQTATFKGTFAEATAEAYRYADLLSKENGKYTADLEDGGYTINIRFAGKEEPEE

SEQ ID NO: 10 - protein, 61 aa, Finegoldia magna
VTIKANLIFANGSTQTAEFKGTFEKATSEAYAYADTLKKDNGEYTVDVADKGYTLNIKFAG

SEQ ID NO: 11 - protein, 61 aa, Finegoldia magna
VTIKANLIYADGKTQTAEFKGTFEEAAAEAYRYADALKKDNGEYTVDVADKGYTLNIKFAG

SEQ ID NO: 12 - protein, 61 aa, Finegoldia magna
VTIKANLIYADGKTQTAEFKGTFEEATAEAYRYADLLAKENGKYTVDVADKGYTLNIKFAG

SEQ ID NO: 13 - protein, 61 aa, Finegoldia magna
VTIKANLIYADGKTQTAEFKGTFAEATAEAYRYADLLAKENGKYTADLEDGGYTINIRFAG

SEQ ID NO: 14 - protein, 61 aa, Finegoldia magna
VTIKENIYFEDGTVQTATFKGTFAEATAEAYRYADLLSKENGKYTADLEDGGYTINIRFAG

SEQ ID NO: 15 - protein, 61 aa, Finegoldia magna
VTIKVNLIFADGKTQTAEFKGTFEEATAKAYAYADLLAKENGEYTADLEDGGNTINIKFAG

SEQ ID NO: 16 - protein, 61 aa, Finegoldia magna
VTIKVNLIFADGKIQTAEFKGTFEEATAKAYAYANLLAKENGEYTADLEDGGNTINIKFAG

SEQ ID NO: 17 - protein, 61 aa, Finegoldia magna
VTIKVNLIFADGKTQTAEFKGTFEEATAEAYRYADLLAKVNGEYTADLEDGGYTINIKFAG

SEQ ID NO: 18 - protein, 61 aa, synthetic construct
VTIKANLIFADGKTQTAEFKGTFEEATAEAYRYADLLAKVNGEYTADLEDGGYTINIKFAG

SEQ ID NO: 19 - protein, 72 aa, Finegoldia magna
PEEEVTIKANLIFADGSTQNAEFKGTFAKAVSDAYAYADALKKDNGEYTVDVADKGLTLNIKFAGKKEKPEE

SEQ ID NO: 20 - protein, 72 aa, synthetic construct
PKEEVTIKAQLIYADGKTQTAEFKGTFEEATAEAYRYADLLAKEAGKYTVDVADKGYTLQIKFAGKEKTPEE

SEQ ID NO: 21 - protein, 72 aa, synthetic construct
PKEEVTIKAHLIYADGKTQTAEFKGTFEEATAEAYRYADLLAKEHGKYTVDVADKGYTLYIKFAGKEKTPEE

SEQ ID NO: 22 - protein, 72 aa, synthetic construct
PKEEVTIKAHLIYADGKTQTAEFKGTFEEATAEAYRYADLLAKEHGKYTVDVADKGYTLQIKFAGKEKTPEE

SEQ ID NO: 23 - protein, 72 aa, synthetic construct
SEEEVTIKAHLIFANGSTQTAEFKGTFEKATSEAYAYADTLKKDHGEYTVDVADKGYTLYIKFAGKEKTPEE

SEQ ID NO: 24 - protein, 72 aa, synthetic construct
SEEEVTIKAHLIFANGSTQTAEFKGTFEKATSEAYAYADTLKKDHGEYTVDVADKGYTLQIKFAGKEKTPEE

SEQ ID NO: 25 - protein, 72 aa, synthetic construct
PKEEVTIKAHLIYADGKTQTAEFKGTFEEAAAEAYRYADALKKDHGEYTVDVADKGYTLYIKFAGKEKTPEE

SEQ ID NO: 26 - protein, 72 aa, synthetic construct
PKEEVTIKAHLIYADGKTQTAEFKGTFEEAAAEAYRYADALKKDHGEYTVDVADKGYTLQIKFAGKEKTPEE

SEQ ID NO: 27 - protein, 74 aa, synthetic construct
PKEEVTIKAHLIYADGKTQTAEFKGTFAEATAEAYRYADLLAKEHGKYTADLEDGGYTIYIRFAGKKVDEKPEE

SEQ ID NO: 28 - protein, 74 aa, synthetic construct
PKEEVTIKAHLIYADGKTQTAEFKGTFAEATAEAYRYADLLAKEHGKYTADLEDGGYTIQIRFAGKKVDEKPEE

SEQ ID NO: 29 - protein, 70 aa, synthetic construct
KEQVTIKEHIYFEDGTVQTATFKGTFAEATAEAYRYADLLSKEHGKYTADLEDGGYTIQIRFAGKEEPEE

SEQ ID NO: 30 - protein, 70 aa, synthetic construct; identical to SEQ ID NO: 29

SEQ ID NO: 31 - protein, 74 aa, synthetic construct
PKEEVTIKVNLIFADGKTQTAEFKGTFEEATAKAYAYADLLAKENGEYTADLEDGGYTINIKFAGKETPETPEE

SEQ ID NO: 32 - protein, 61 aa, synthetic construct
VTIKVNLIFADGKTQTAEFKGTFEEATAKAYAYADLLAKENGEYTADLEDGGYTINIKFAG

SEQ ID NO: 33 - protein, 74 aa, synthetic construct
PKEEVTIKVNLIFADGKIQTAEFKGTFEEATAKAYAYADLLAKENGEYTADLEDGGYTINIKFAGKETPETPEE

SEQ ID NO: 34 - protein, 61 aa, synthetic construct
VTIKVNLIFADGKIQTAEFKGTFEEATAKAYAYADLLAKENGEYTADLEDGGYTINIKFAG

SEQ ID NO: 35 - protein, 74 aa, synthetic construct
PKEEVTIKVHLIFADGKTQTAEFKGTFEEATAKAYAYADLLAKEHGEYTADLEDGGYTIYIKFAGKETPETPEE

SEQ ID NO: 36 - protein, 74 aa, synthetic construct
PKEEVTIKVHLIFADGKTQTAEFKGTFEEATAKAYAYADLLAKEHGEYTADLEDGGYTIQIKFAGKETPETPEE

SEQ ID NO: 37 - protein, 74 aa, synthetic construct
PKEEVTIKVHLIFADGKIQTAEFKGTFEEATAKAYAYADLLAKEHGEYTADLEDGGYTIYIKFAGKETPETPEE

SEQ ID NO: 38 - protein, 74 aa, synthetic construct
PKEEVTIKVHLIFADGKIQTAEFKGTFEEATAKAYAYADLLAKEHGEYTADLEDGGYTIQIKFAGKETPETPEE

SEQ ID NO: 39 - protein, 74 aa, synthetic construct
PKEEVTIKVHLIFADGKTQTAEFKGTFEEATAEAYRYADLLAKVHGEYTADLEDGGYTIYIKFAGKEQPGENPG

SEQ ID NO: 40 - protein, 74 aa, synthetic construct
PKEEVTIKVHLIFADGKTQTAEFKGTFEEATAEAYRYADLLAKVHGEYTADLEDGGYTIQIKFAGKEQPGENPG

SEQ ID NO: 41 - protein, 72 aa, synthetic construct
PKEEVTIKAHLIFADGKTQTAEFKGTFEEATAEAYRYADLLAKVHGEYTADLEDGGYTIYIKFAGKEQPGEN

SEQ ID NO: 42 - protein, 72 aa, synthetic construct
PKEEVTIKAHLIFADGKTQTAEFKGTFEEATAEAYRYADLLAKVHGEYTADLEDGGYTIQIKFAGKEQPGEN

SEQ ID NO: 43 - protein, 61 aa, synthetic construct
VTIKAHLIFANGSTQTAEFKGTFEKATSEAYAYADTLKKDHGEYTVDVADKGYTLYIKFAG

SEQ ID NO: 44 - protein, 61 aa, synthetic construct
VTIKAHLIFANGSTQTAEFKGTFEKATSEAYAYADTLKKDHGEYTVDVADKGYTLQIKFAG

SEQ ID NO: 45 - protein, 61 aa, synthetic construct
VTIKAHLIYADGKTQTAEFKGTFEEAAAEAYRYADALKKDHGEYTVDVADKGYTLYIKFAG

SEQ ID NO: 46 - protein, 61 aa, synthetic construct
VTIKAHLIYADGKTQTAEFKGTFEEAAAEAYRYADALKKDHGEYTVDVADKGYTLQIKFAG

SEQ ID NO: 47 - protein, 61 aa, synthetic construct
VTIKAHLIYADGKTQTAEFKGTFEEATAEAYRYADLLAKEHGKYTVDVADKGYTLYIKFAG

SEQ ID NO: 48 - protein, 61 aa, synthetic construct
VTIKAHLIYADGKTQTAEFKGTFEEATAEAYRYADLLAKEHGKYTVDVADKGYTLQIKFAG

SEQ ID NO: 49 - protein, 61 aa, synthetic construct
VTIKAHLIYADGKTQTAEFKGTFAEATAEAYRYADLLAKEHGKYTADLEDGGYTIYIRFAG

SEQ ID NO: 50 - protein, 61 aa, synthetic construct
VTIKAHLIYADGKTQTAEFKGTFAEATAEAYRYADLLAKEHGKYTADLEDGGYTIQIRFAG

SEQ ID NO: 51 - protein, 61 aa, synthetic construct
VTIKEHIYFEDGTVQTATFKGTFAEATAEAYRYADLLSKEHGKYTADLEDGGYTIYIRFAG

SEQ ID NO: 52 - protein, 61 aa, synthetic construct
VTIKEHIYFEDGTVQTATFKGTFAEATAEAYRYADLLSKEHGKYTADLEDGGYTIQIRFAG

SEQ ID NO: 53 - protein, 61 aa, synthetic construct
VTIKVHLIFADGKTQTAEFKGTFEEATAKAYAYADLLAKEHGEYTADLEDGGYTIYIKFAG

SEQ ID NO: 54 - protein, 61 aa, synthetic construct
VTIKVHLIFADGKTQTAEFKGTFEEATAKAYAYADLLAKEHGEYTADLEDGGYTIQIKFAG

SEQ ID NO: 55 - protein, 61 aa, synthetic construct
VTIKVHLIFADGKIQTAEFKGTFEEATAKAYAYADLLAKEHGEYTADLEDGGYTIYIKFAG

SEQ ID NO: 56 - protein, 61 aa, synthetic construct
VTIKVHLIFADGKIQTAEFKGTFEEATAKAYAYADLLAKEHGEYTADLEDGGYTIQIKFAG

SEQ ID NO: 57 - protein, 61 aa, synthetic construct
VTIKVHLIFADGKTQTAEFKGTFEEATAEAYRYADLLAKVHGEYTADLEDGGYTIYIKFAG

SEQ ID NO: 58 - protein, 61 aa, synthetic construct
VTIKVHLIFADGKTQTAEFKGTFEEATAEAYRYADLLAKVHGEYTADLEDGGYTIQIKFAG

SEQ ID NO: 59 - protein, 61 aa, synthetic construct
VTIKAHLIFADGKTQTAEFKGTFEEATAEAYRYADLLAKVHGEYTADLEDGGYTIYIKFAG

SEQ ID NO: 60 - protein, 61 aa, synthetic construct
VTIKAHLIFADGKTQTAEFKGTFEEATAEAYRYADLLAKVHGEYTADLEDGGYTIQIKFAG

(19) United States
(12) Patent Application Publication
(10) Pub. No.: US 2024/0391984 A1
(43) Pub. Date: Nov. 28, 2024

(54) MODIFIED KAPPA LIGHT CHAIN-BINDING POLYPEPTIDES

(71) Applicant: Cytiva Bioprocess R&D AB, Uppsala (SE)

(72) Inventors: Mats Ander, Uppsala (SE); Joakim Galli, Uppsala (SE); Andreas L. Jonsson, Uppsala (SE); Gustav Rodrigo, Uppsala (SE)

(21) Appl. No.: 18/682,409

(22) PCT Filed: Aug. 24, 2022
(86) PCT No.: PCT/EP2022/073599

§ 371 Date: Feb. 8, 2024

(30) Foreign Application Priority Data
Aug. 31, 2021 (GB) 2112386.4

(51) Int. Cl. C07K 16/12; C07K 1/22

(52) U.S. Cl. CPC C07K 16/1282; C07K 1/22

(57) ABSTRACT

The present relates to a polypeptide that binds to an immunoglobulin or a fragment thereof. More specifically, it relates to a kappa light-chain binding polypeptide with high binding affinity and improved alkali stability. The one kappa light-chain binding comprises a mutated binding domain of Peptostreptococcus Protein L, derived from any one of the amino acid sequences SEQ ID NO:10, SEQ ID NO:11, SEQ ID NO:12, SEQ ID NO:13, SEQ ID NO:14, SEQ ID NO:15, SEQ ID NO:16, SEQ ID NO:17 or SEQ ID NO:18, said amino acid sequences having N6H, N41H and N56Y or N56Q mutations.

### TECHNICAL FIELD

The present relates to a polypeptide that binds to an immunoglobulin or a fragment thereof. More specifically, it relates to a kappa light-chain binding polypeptide with high binding affinity and improved alkali stability.

### BACKGROUND

Immunoglobulins and immunoglobulin fragments represent the most prevalent biopharmaceutical products in either manufacture or development worldwide. The high commercial demand for and hence value of this particular therapeutic market has led to the emphasis being placed on pharmaceutical companies to maximize the productivity of their respective manufacturing processes whilst controlling the associated costs.

Affinity chromatography, typically on matrices comprising staphylococcal Protein A or variants thereof, is normally used as one of the key steps in the purification of intact immunoglobulin molecules. The highly selective binding of Protein A to the Fc chain of immunoglobulins provides for a generic step with very high clearance of impurities and contaminants.

For antibody fragments, such as Fab, single-chain variable fragments (scFv), bi-specific T-cell engagers (BiTEs), domain antibodies etc., which lack the Fc chain but have a kappa light chain subclass 1, 3 or 4, matrices comprising Protein L derived from Peptostreptococcus magnus (B Äkerström, L Björck: J. Biol. Chem. 264, 19740-19746, 1989; W Kastern et al: J. Biol. Chem. 267, 12820-12825, 1992; B HK Nilson et al: J. Biol. Chem. 267, 2234-2239, 1992 and 30 U.S. Pat. No. 6,822,075) are used as a purification platform providing the high selectivity needed.

Protein L matrices are commercially available as for instance Capto™ L from Cytiva™ and can be used for separation of kappa light chain-containing proteins such as intact antibodies, Fab fragments, scFv fragments, domain antibodies etc. About 75% of the antibodies produced by healthy humans have a kappa light chain and about 90% of therapeutic monoclonal antibodies and antibody fragments contain kappa light chains (Carter, P., Lazar, G. Next generation antibody drugs: pursuit of the ‘high-hanging fruit’. Nat Rev Drug Discov 17, 197-223 (2018). https://doi.org/10.1038/nrd.2017.227).

Protein L is a 76-106 kDa protein containing four or five highly homologous, consecutive extracellular Ig binding domains, depending on the bacterial strain from which it is isolated. The gene for Protein L in Peptostreptococcus magnus strain 312 contains several components, including a signal sequence of 18 AA, an amino terminal region of 90 AA, followed by 5 homologous antibody binding domains. In the Capto™ L product, the ligand consists of the functional domains B1-B4 (WO 00/15803 A1). Moreover, Peptostreptococcus magnus strain 3316 produces a homologous protein L, consisting of four IgG binding domains named C1-C4. An additional homologous protein has been found in GenBank database, considered “hypothetical protein”, herein called D domains.

Any bioprocess chromatography application requires comprehensive attention to definite removal of contaminants. Such contaminants can for example be non-eluted molecules adsorbed to the stationary phase or matrix in a chromatographic procedure, such as non-desired biomolecules or microorganisms, including for example proteins, carbohydrates, lipids, bacteria and viruses. The removal of such contaminants from the matrix is usually performed after a first elution of the desired product in order to regenerate the matrix before subsequent use. Such removal usually involves a procedure known as cleaning-in-place (CIP), wherein agents capable of eluting contaminants from the stationary phase are used. One such class of agents often used with chromatography media is alkaline solutions that are passed over the matrix. At present the most extensively used cleaning and sanitizing agent is NaOH, and it is desirable to use it in concentrations ranging from 0.05 up to e.g. 1 M, depending on the degree and nature of contamination. Protein L is however a rather alkali-sensitive protein compared to e.g. Protein A and only tolerates 15 mM NaOH for 80 cycles using 15 min contact time with 90% remaining binding capacity. Due to capacity loss of the resin additional, less desirable cleaning solutions, e.g. urea or guanidinium salts, may also have to be used in order to ensure sufficient cleaning.

There is thus a need in this field to obtain a separation matrix containing Protein L-derived ligands having an improved stability towards alkaline cleaning procedures.

### SUMMARY OF THE INVENTION

It has been an objective for the inventors to provide a Protein L ligand with a high alkali stability. It has further been an objective for the inventors to provide a Protein L ligand with at least maintained affinity and selectivity.

The objective has been attained by providing a kappa light chain-binding polypeptide consisting of, consisting essentially of, or comprising at least one mutated binding domain of Peptostreptococcus Protein L, which domain has at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:10, SEQ ID NO:11, SEQ ID NO:12, SEQ ID NO:13, SEQ ID NO:14, SEQ ID NO:15, SEQ ID NO:16, SEQ ID NO:17 or SEQ ID NO:18, and wherein the polypeptide has the asparagines in each of the positions 6 and 41 mutated to a histidine, and the asparagine in position 56 mutated to a tyrosine or a glutamine relative to any one of SEQ ID NO:s 10-18.

The binding domain of Peptostreptococcus Protein L may have at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:1, SEQ ID NO:2, SEQ ID NO:3, SEQ ID NO: 4, SEQ ID NO: 5, SEQ ID NO:6, SEQ ID NO:7 or SEQ ID NO:8, and wherein the polypeptide has the asparagines in each of the positions 10 and 45 mutated to a histidine, and the asparagine in position 60 mutated to a tyrosine or a glutamine relative to SEQ ID NO: 1-4 and 6-9; or
at least 90%, 95% or 98% sequence identity, or a 77.5% sequence similarity as determined by BLOSUM matrix of 75, with a gap open penalty of 12, a gap extension penalty of 3, with SEQ ID NO: 9 and wherein the polypeptide has the asparagines in each of the positions 9 and 44 mutated to a histidine, and the asparagine in position 59 mutated to a tyrosine or a glutamine relative to SEQ ID NO: 5.

The binding domain of Peptostreptococcus Protein L may be selected from the group comprising of a B2 domain, a B3 domain, a B4 domain, a C2 domain, a C3 domain, a C4 domain and a D1 domain. Preferably, the binding domain of Peptostreptococcus Protein L is selected from the group comprising of the B3 domain, the C2 domain, the C3 domain and the D-domain.

The domain may have a 90%, 95% or 98% sequence identity, or a 77.5% sequence similarity as determined by BLOSUM matrix of 75, with a gap open penalty of 12, a gap extension penalty of 3, with any one of the sequences SEQ ID NO:3, SEQ ID NO:6 or SEQ ID NO:7.

The C2 domain may be a domain wherein, additionally, the asparagine in position 57 has been mutated to a tyrosine or a glutamine, such as a tyrosine. The domain may have a 90%, 95% or 98% sequence identity, or a 77.5% sequence similarity as determined by BLOSUM matrix of 75, with a gap open penalty of 12, a gap extension penalty of 3, with SEQ ID NO:31 or SEQ ID NO: 32.

The C3 domain may be a domain wherein, additionally, the asparagine in position 57 has been mutated to a tyrosine or a glutamine, such as a tyrosine, and an asparagine in position 39 has been mutated to an aspartic acid. The domain may have a 90%, 95% or 98% sequence identity, or a 77.5% sequence similarity as determined by BLOSUM matrix of 75, with a gap open penalty of 12, a gap extension penalty of 3, with SEQ ID NO:33 or SEQ ID NO: 34.

The polypeptide may have a 90%, 95% or 98% sequence identity, or a 77.5% sequence similarity as determined by BLOSUM matrix of 75, with a gap open penalty of 12, a gap extension penalty of 3, with any one of the sequences SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:41, SEQ ID NO:52, SEQ ID NO:53, SEQ ID NO:54, SEQ ID NO:55, SEQ ID NO:56, SEQ ID NO:57, SEQ ID NO:58, SEQ ID NO:59 or SEQ ID NO:60.

Preferably, the polypeptide has a 90%, 95% or 98% sequence identity, or a 77.5% sequence similarity as determined by BLOSUM matrix of 75, with a gap open penalty of 12, a gap extension penalty of 3, with any one of the sequences SEQ ID NO:21, SEQ ID NO:22, SEQ ID NO:35, SEQ ID NO:36, SEQ ID NO:37, SEQ ID NO:38, SEQ ID NO:47, SEQ ID NO:48, SEQ ID NO:53, SEQ ID NO:54, SEQ ID NO:55 or SEQ ID NO:56, such as with any one of the sequences SEQ ID NO:21, SEQ ID NO:22, SEQ ID NO:47 or SEQ ID NO:48.

The kappa light chain-binding polypeptide may further comprise a spacer or a linker N-terminally or C-terminally of the specified amino acid sequence, and/or additional amino acid(s) N-terminally or C-terminally of the specified amino acid sequence. The kappa light chain-binding polypeptide may further comprise, at the N-terminus, a plurality of amino acid residues originating from the cloning process or constituting a residue from a cleaved off signaling sequence, wherein the number of additional amino acid residues is 15 or less, such as 10 or less or 5 or less.

The kappa light chain-binding polypeptide preferably binds to κ1, κ3 and κ4.

Also provided herein is a multimer comprising at least two of the polypeptides according to any one of claims 1-15, such as two, three, four, five, six, seven, eight or nine polypeptides. The multimer may further comprise a linker, spacer, or additional amino acid(s).

Further provided herein is a nucleic acid encoding the polypeptide or multimer according to the above.

Additionally provided is a vector comprising the nucleic acid according to the above, optionally further comprising one or more of a signal peptide, enhancer, promotor, identification tag, identification marker, selection marker, and/or purification tag.

The present disclosure also provides for an expression system comprising the nucleic acid or the vector according to the above.

Additionally, the present disclosure provides for a separation matrix comprising at least one polypeptide, or at least one multimer according to the above, coupled to a solid support.

Preferred aspects of the present disclosure are described below in the detailed description and in the dependent claims.

### Definitions

The terms “antibody” and “immunoglobulin” are used interchangeably herein, and are understood to include also fragments of antibodies, fusion proteins comprising antibodies or antibody fragments and conjugates comprising antibodies or antibody fragments.

The terms a “kappa light chain-binding polypeptide” and “kappa light chain-binding protein” herein mean a polypeptide or protein respectively, capable of binding to a subclass 1, 3 or 4 kappa light chain of an antibody (also called V_{κI}, V_{κIII} and V_{κIV}, as in B HK Nilson et al: J. Biol. Chem. 267, 2234-2239, 1992), and include e.g. Protein L, and any variant, fragment or fusion protein thereof that has maintained said binding property.

The term “kappa light chain-containing protein” is used as a synonym of “immunoglobulin kappa light chain-containing protein” and herein means a protein comprising a subclass 1, 3 or 4 kappa light chain (also called V_{κI}, V_{κIII} and V_{κIV}, as in B HK Nilson et al: J. Biol. Chem. 267, 10 2234-2239, 1992) derived from an antibody and includes any intact antibodies, antibody fragments, fusion proteins, conjugates or recombinant proteins containing a subclass 1, 3 or 4 kappa light chain.

The term “linker” herein means an element linking two polypeptide units, monomers or domains to each other in a multimer.

The term “spacer” herein means an element connecting a polypeptide or a polypeptide multimer to a support.

### DRAWINGS

FIG. 1. Alignment of Protein L kappa light chain-binding domains, full-length and truncated.

FIG. 2. NaOH stability of protein L ligands, B3 domain.

FIG. 3. Elution profiles for Protein L Ligands, B3 domain. Normalized Value of Ligand Bound.

FIG. 4. NaOH stability of domains B2, B3, B4, C2b, C3b, C4 and D1 over 100 cycles, 0.1 M NaOH.

FIG. 5. Elution profile of B2, B3, B4, C2, C2b, C3b, C4 and D1. HHQ variants have white marker, HHY variants have black marker.

FIG. 6. Blosum75 analysis. A) alignment of sequences. B) Table over distances similarity.

### DETAILED DESCRIPTION OF THE INVENTION

While there are Protein L separation matrices available on the market, there is still a need for further improvement. In particular there is a need to improve the alkali stability thereof. Thus, there is a need for matrices having excellent stability under alkaline conditions. It has thus been an objective for the inventors to produce a Protein L ligand with higher alkali stability and at least maintained affinity towards κ1, κ3 and κ4 light chains, as compared to existing Protein L ligands.

The kappa light chain-binding domains of Protein L which are of interest have the wildtype (wt) backbone sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO:8 and SEQ ID NO: 9.

(wt B1)
SEQ ID NO: 1
SEEEVTIKANLIFANGSTQTAEFKGTFEKATSEAYAYADTLKKDNGEYT
VDVADKGYTLNIKFAGKEKTPEE
(wt B2)
SEQ ID NO: 2
PKEEVTIKANLIYADGKTQTAEFKGTFEEAAAEAYRYADALKKDNGEYT
VDVADKGYTLNIKFAGKEKTPEE
(wt B3)
SEQ ID NO: 3
PKEEVTIKANLIYADGKTQTAEFKGTFEEATAEAYRYADLLAKENGKYT
VDVADKGYTLNIKFAGKEKTPEE
(wt B4)
SEQ ID NO: 4
PKEEVTIKANLIYADGKTQTAEFKGTFAEATAEAYRYADLLAKENGKYT
ADLEDGGYTINIRFAGKKVDEKPEE
(wt C2)
SEQ ID NO: 5
PKEEVTIKVNLIFADGKTQTAEFKGTFEEATAKAYAYADLLAKENGEYT
ADLEDGGNTINIKFAGKETPETPEE
(wt C3)
SEQ ID NO: 6
PKEEVTIKVNLIFADGKIQTAEFKGTFEEATAKAYAYANLLAKENGEYT
ADLEDGGNTINIKFAGKETPETPEE
(wt C4)
SEQ ID NO: 7
PKEEVTIKVNLIFADGKTQTAEFKGTFEEATAEAYRYADLLAKVNGEYT
ADLEDGGYTINIKFAGKEQPGENPG
(D1)
SEQ ID NO: 8
PKEEVTIKANLIFADGKTQTAEFKGTFEEATAEAYRYADLLAKVNGEYT
ADLEDGGYTINIKFAGKEQPGEN

The B5 domain used in the present disclosure has an H45N mutation, and hence have the backbone sequence according to SEQ ID NO: 9. This was done to ensure that there were at least three asparagines in all the backbone sequences tested.

(B5)
SEQ ID NO 9
KEQVTIKENIYFEDGTVQTATFKGTFAEATAEAYRYADLLSKENGKYTA
DLEDGGYTINIRFAGKEEPEE

B1-B4, and C2-C4 has been previously published in for instance U.S. Pat. No. 6,162,903, WO 00/15803 A1 and J. P. Murphy et al. (Mol. Microbiol. (1994) 12, 911-920), as well as B5 without the H45N mutation. D1 is the inventors' nomenclature for a sequence that has been previously published as WP_094225182.1 in NCBI database, amino acids 548-619.

The three-dimensional structure of Protein L, starting from the N-terminal, comprises two beta sheets, one alpha helix, and two additional beta sheets (see for instance Graille, M. et al., Structure 2001 (9), p. 679-687). Positions 1-4 precede the first beta sheet which starts at position 5. The fourth beta sheet ends at position 65, thus position 66 and any following amino acid positions do not form part of the above-mentioned three-dimensional structures.

Thus, sequences SEQ ID NO:1-9 can alternatively be written as truncated sequences, with amino acids in positions 1-4 omitted as well as any amino acids following position 65 also omitted. Thus, an alternative language for disclosing Sequences SEQ ID NO:s 1-9 is that the kappa light chain-binding domain of Protein L is an amino acid sequence according to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18.

(wt B1_trunc)
SEQ ID NO: 10
----VTIKANLIFANGSTQTAEFKGTFEKATSEAYAYADTLKKDNGEYT
VDVADKGYTLNIKFAG-------
(wt B2_trunc)
SEQ ID NO: 11
----VTIKANLIYADGKTQTAEFKGTFEEAAAEAYRYADALKKDNGEYT
VDVADKGYTLNIKFAG-------
(wt B3_trunc)
SEQ ID NO: 12
----VTIKANLIYADGKTQTAEFKGTFEEATAEAYRYADLLAKENGKYT
VDVADKGYTLNIKFAG-------
(wt B4_trunc)
SEQ ID NO: 13
----VTIKANLIYADGKTQTAEFKGTFAEATAEAYRYADLLAKENGKYT
ADLEDGGYTINIRFAG---------
(B5_trunc)
SEQ ID NO 14
----VTIKENIYFEDGTVQTATFKGTFAEATAEAYRYADLLSKENGKYT
ADLEDGGYTINIRFAG------
(wt C2_trunc)
SEQ ID NO: 15
----VTIKVNLIFADGKTQTAEFKGTFEEATAKAYAYADLLAKENGEYT
ADLEDGGNTINIKFAG---------
(wt C3_trunc)
SEQ ID NO: 16
----VTIKVNLIFADGKIQTAEFKGTFEEATAKAYAYANLLAKENGEYT
ADLEDGGNTINIKFAG---------
(wt C4_trunc)
SEQ ID NO: 17
----VTIKVNLIFADGKTQTAEFKGTFEEATAEAYRYADLLAKVNGEYT
ADLEDGGYTINIKFAG---------
(D1_trunc)
SEQ ID NO: 18
----VTIKANLIFADGKTQTAEFKGTFEEATAEAYRYADLLAKVNGEYT
ADLEDGGYTINIKFAG-------

The inventors have now determined key positions and mutations in a Protein L domain, in order to improve the alkali stability while maintaining the binding affinity for a Protein L ligand. Thereby, the above-mentioned objective has been attained by developing a kappa light chain-binding polypeptide consisting of, consisting essentially of, or comprising at least one mutated kappa light chain-binding domain of Peptostreptococcus Protein L
having at least 90%, 95% or 98% sequence identity or a 77.5% sequence similarity as determined by BLOSUM matrix of 75, with a gap open penalty of 12, a gap extension penalty of 3, with any one of the amino acid sequences SEQ ID NO:1, SEQ ID NO:2, SEQ ID NO:3, SEQ ID NO: 4, SEQ ID NO: 5, SEQ ID NO:6, SEQ ID NO:7 or SEQ ID NO:8, and wherein the polypeptide has the asparagines (N) in each of the positions 10 and 45 mutated to a histidine (H), and the asparagine (N) in position 60 mutated to a tyrosine (Y) or a glutamine (Q) relative to SEQ ID NO: 1-8; or
having at least 90%, 95% or 98% sequence identity, or a 77.5% sequence similarity as determined by BLOSUM matrix of 75, with a gap open penalty of 12, a gap extension penalty of 3, with SEQ ID NO: 9, and wherein the polypeptide has the asparagines (N) in each of the positions 9 and 44 mutated to a histidine (H), and the asparagine (N) in position 59 mutated to a tyrosine (Y) or a glutamine (Q) relative to SEQ ID NO: 5.

In an alternative language, the invention relates to a kappa light chain-binding polypeptide consisting of, consisting essentially of, or comprising at least one mutated binding domain of Peptostreptococcus Protein L
having at least 90%, 95% or 98% sequence identity, or a 77.5% sequence similarity as determined by BLOSUM matrix of 75, with a gap open penalty of 12, a gap extension penalty of 3, with any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17 or SEQ ID NO:18,
and wherein the polypeptide has the asparagines (N) in each of the positions 6 and 41 mutated to a histidine (H), and the asparagine (N) in position 56 mutated to a tyrosine (Y) or a glutamine (Q) relative to any one of SEQ ID NO:s 10-18.

There is one additional Protein L domain that has been previously disclosed, domain C1 (SEQ ID NO: 19). However, this domain is not included in the scope due to the experimental results.

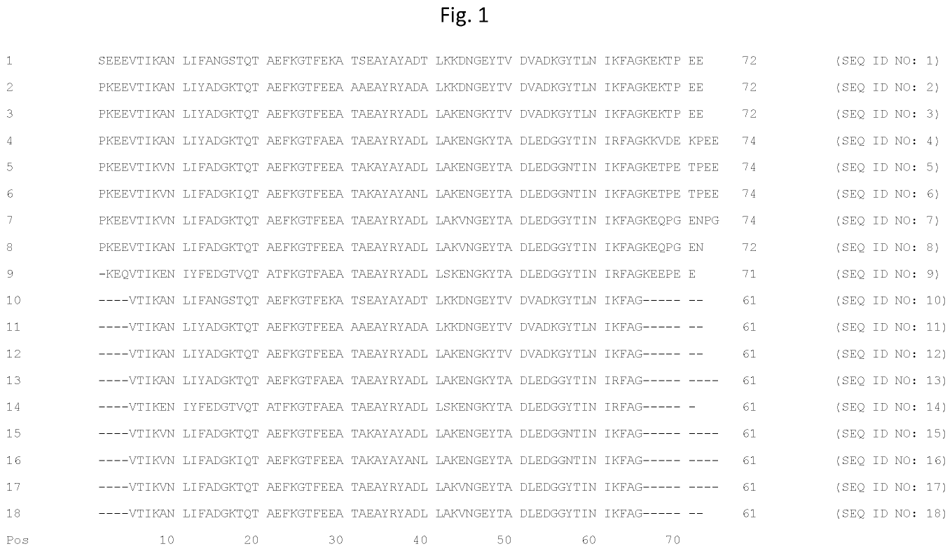

For simplicity, when the description states the positions 10, 45 and 60, it relates to a position for the aligned sequences 1-9. It will thus also encompass the corresponding positions in any truncated sequence. For instance, B5 (SEQ ID NO: 9) has the position corresponding to position 1 in SEQ ID NO:s 1-8 deleted. Thus, it should be clear that the positions 10, 45 and 60 in SEQ ID NO:s 1-8, in an alignment, corresponds to positions 9, 44 and 59, respectively, in SEQ ID NO:9. Conversely, for the truncated sequences, SEQ ID NO:s 10-18, the positions 10, 45 and 60 in SEQ ID NO:s 1-8, in an alignment, corresponds to positions 6, 41 and 56, respectively, in SEQ ID NO:10-18. An alignment of full-length sequence and truncated sequences is shown in FIG. 1. Any specific amino acid sequences explicitly disclosed have the mutations specified per that particular sequence. However, In the text below and in the experimental part, only positions 10, 45 and 60 are referred to.

The reference to sequence identity or sequence similarity, refers to the identity or similarity to the identified sequences, prior to incorporating the specific mutations in positions 10, 45 and 60 as specified above. Thus, the specified mutations in positions 10, 45 and 60 must always be present in the polypeptides according to the present invention. For any variation that falls within the range for identity or similarity, the variation may not apply to these particular positions 10, 45 and 60.

The term “% identity” with respect to comparisons of amino acid sequences is determined by standard alignment algorithms such as, for example, Basic Local Alignment Tool (BLAST™) described in Altshul et al. (1990) J. Mol. Biol., 215: 403-410. A web-based software for this is freely available from the US National Library of Medicine at http://blast.ncbi.nlm.nih.gov/Blast.cgi?PROGRAM=blastp&PAGE_TYPE=BlastSearch&LINK_LOC=blasthome. Here, the algorithm “blastp (protein-protein BLAST)” is used for alignment of a query sequence with a subject sequence and determining i.a. the % identity.

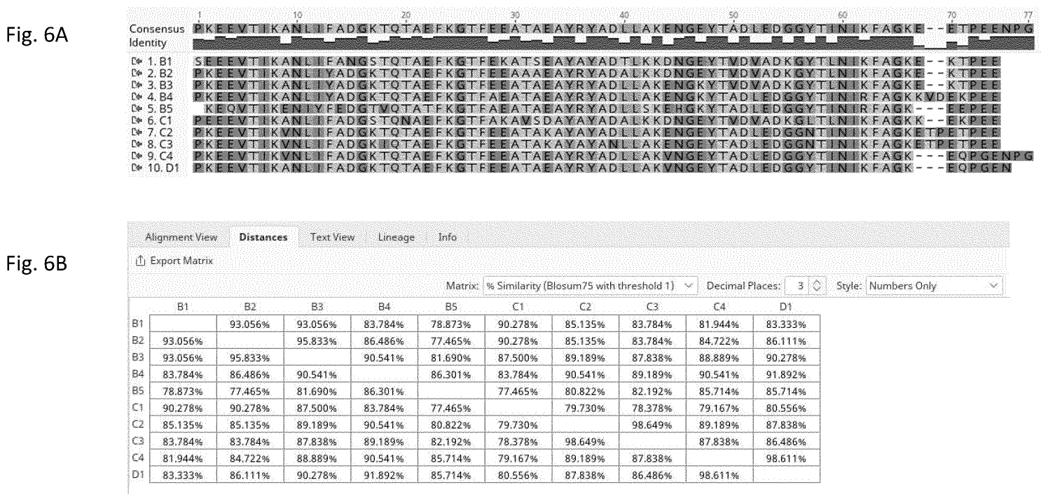

The term “similarity” with respect to comparisons of amino acid sequences is determined by standard alignment algorithms such as, for example, Geneious Prime software (available from https://www.geneious.com/) Herein, the Geneious Alignment tool was used for a query on ten Protein L sequences, SEQ ID NO:s 1-9 and SEQ ID NO:19, with a global alignment with free end gaps using a Blosum75 matrix with a gap open penalty of 12, a gap extension penalty of 3, and refinement iterations of 2. The alignment is shown in FIG. 6a. The result of the distance similarity is shown in FIG. 6b.

The polypeptide may further comprise 3-5, such as 4, amino acids N-terminally of the above-mentioned truncated sequences SEQ ID NO: 10-18. The polypeptide may further comprise 5-10, such as 6, 7, 8 or 9 amino acids C-terminally of the above-mentioned truncated sequences SEQ ID NO: 10-18. These additional amino acids may differ from those present at the corresponding positions in any of the amino acid sequences SED ID NO: 1-9.

The polypeptide may further at the N-terminus comprise a plurality of amino acid residues originating from the cloning process or constituting a residue from a cleaved off signaling sequence. The number of additional amino acid residues may e.g. be 15 or less, such as 10 or less or 5 or less.

The polypeptide may further comprise a spacer or a linker N-terminally or C-terminally of the specified amino acid sequence, and/or additional amino acid(s) N-terminally or C-terminally of the specified amino acid sequence. Said spacer, linker and/or additional amino acid(s) are in general not involved in the kappa light chain-binding function.

A previously disclosed Protein L ligand (WO 2016/096644) corresponds to SEQ ID NO.20.

SEQ ID NO: 20
PKEEVTIKAQLIYADGKTQTAEFKGTFEEATAEAYRYADLLAKEAGKYT
VDVADKGYTLQIKFAGKEKTPEE

The previously disclosed sequence above (SEQ ID NO:20) was used as a benchmarking sequence in the experiments disclosed below, as was the wtB3 domain (SEQ ID NO: 3).

According to one embodiment, the kappa light chain-binding polypeptide has an amino acid sequence selected from the group comprising the sequences defined by any one of SEQ ID NO: 21, SEQ ID NO:22, SEQ ID NO:23, SEQ ID NO:24, SEQ ID NO:25, SEQ ID NO:26, SEQ ID NO:27, SEQ ID NO:28, SEQ ID NO:29 and SEQ ID NO:30.

(B3: N10H, N45H, N60Y mutations)
SEQ ID NO: 21
PKEEVTIKAH LIYADGKTQT AEFKGTFEEA TAEAYRYADL
LAKEHGKYTV DVADKGYTLY IKFAGKEKTP EE
(B3: N10H, N45H, N60Q mutations)
SEQ ID NO: 22
PKEEVTIKAH LIYADGKTQT AEFKGTFEEA TAEAYRYADL
LAKEHGKYTV DVADKGYTLQ IKFAGKEKTP EE
(B1: N10H, N45H, N60Y mutations)
SEQ ID NO: 23
SEEEVTIKAHLIFANGSTQTAEFKGTFEKATSEAYAYADTLKKDHGEYT
VDVADKGYTLYIKFAGKEKTPEE
(B1: N10H, N45H, N60Q mutations)
SEQ ID NO: 24
SEEEVTIKAHLIFANGSTQTAEFKGTFEKATSEAYAYADTLKKDHGEYT
VDVADKGYTLQIKFAGKEKTPEE
(B2: N10H, N45H, N60Y mutations)
SEQ ID NO: 25
PKEEVTIKAHLIYADGKTQTAEFKGTFEEAAAEAYRYADALKKDHGEYT
VDVADKGYTLYIKFAGKEKTPEE
(B2: N10H, N45H, N60Q mutations)
SEQ ID NO: 26
PKEEVTIKAHLIYADGKTQTAEFKGTFEEAAAEAYRYADALKKDHGEYT
VDVADKGYTLQIKFAGKEKTPEE
SEQ ID NO: 27(B4: N10H, N45H, N60Y mutations)
PKEEVTIKAHLIYADGKTQTAEFKGTFAEATAEAYRYADLLAKEHGKYT
ADLEDGGYTIYIRFAGKKVDEKPEE
SEQ ID NO: 28(B4: N10H, N45H, N60Q mutations)
PKEEVTIKAHLIYADGKTQTAEFKGTFAEATAEAYRYADLLAKEHGKYT
ADLEDGGYTIQIRFAGKKVDEKPEE
(B5: N9H, N44H, N59Y mutations)
SEQ ID NO 29
KEQVTIKEH IYFEDGTVQTATFKGTFAEATAEAYRYADLLSKEHGKYT
ADLEDGGYTIQIRFAGKEEPEE
(B5: N9H, N44H, N59Q mutations)
SEQ ID NO 30
KEQVTIKEHIYFEDGTVQTATFKGTFAEATAEAYRYADLLSKEHGKYTA
DLEDGGYTIQIRFAGKEEPEE

For the C2-domain, it was found that the wt backbone (SEQ ID NO: 5) has an unsatisfactory alkali stability. Therefore, the C2-backbone was mutated to a C2b-domain, SEQ ID NO: 31, wherein an asparagine in position 57 has been mutated to a tyrosine (Y).

(C2b)(C2, N57Y)
SEQ ID NO: 31
PKEEVTIKVNLIFADGKTQTAEFKGTFEEATAKAYAYADLLAKENGEYT
ADLEDGGYTINIKFAGKETPETPEE

The same mutations should be made to any truncated versions, in the corresponding positions upon an alignment with the above SEQ ID NO:31. An example of such a truncated sequence is SEQ ID NO: 32.

(C2b_trunc)
SEQ ID NO: 32
----VTIKVNLIFADGKTQTAEFKGTFEEATAKAYAYADLLAKENGEYT
ADLEDGGYTINIKFAG---------

Thus, according to one embodiment, the mutated kappa light chain-binding domain of Peptostreptococcus Protein L has at least 90%, 95% or 98% sequence identity, or a 77.5% sequence similarity as determined by BLOSUM matrix of 75, with a gap open penalty of 12, a gap extension penalty of 3, with the amino acid sequence SEQ ID NO:31, and wherein the polypeptide has the asparagines (N) in each of the positions 10 and 45 mutated to a histidine (H), and the asparagine (N) in position 60 mutated to a tyrosine (Y) or a glutamine (Q) relative to SEQ ID NO:31.

Alternatively, according to one embodiment, the kappa light chain-binding domain of Peptostreptococcus Protein L has at least 90%, 95% or 98% sequence identity, or a 77.5% sequence similarity as determined by BLOSUM matrix of 75, with a gap open penalty of 12, a gap extension penalty of 3, with amino acid sequence SEQ ID NO:32, and wherein the polypeptide has the asparagines (N) in each of the positions 6 and 41 mutated to a histidine (H), and the asparagine (N) in position 56 mutated to a tyrosine (Y) or a glutamine (Q) relative to SEQ ID NO:32.

Similarly, for the C3-domain, it was found that the wt backbone (SEQ ID NO: 7) has an unsatisfactory alkali stability. Therefore, the C3-backbone was mutated to a C3b-domain, SEQ ID NO:33, wherein an asparagine (N) in position 57 has been mutated to a tyrosine (Y), and an asparagine (N) in position 39 has been mutated to an aspartic acid (D).

(C3b) (C3, N39D, N57Y)
SEQ ID NO: 33
PKEEVTIKVNLIFADGKIQTAEFKGTFEEATAKAYAYADLLAKENGEYT
ADLEDGGYTINIKFAGKETPETPEE

The same mutations should be made to any truncated versions, in the corresponding positions upon an alignment with the above SEQ ID NO:33. An example of such a truncated sequence is SEQ ID NO: 34

(C3b_trunc)
SEQ ID NO: 34
----VTIKVNLIFADGKIQTAEFKGTFEEATAKAYAYADLLAKENGEYT
ADLEDGGYTINIKFAG

Thus, according to one embodiment, the kappa light chain-binding domain of Peptostreptococcus Protein L has at least 90%, 95% or 98% sequence identity, or a 77.5% sequence similarity as determined by BLOSUM matrix of 75, with a gap open penalty of 12, a gap extension penalty of 3, with the amino acid sequence SEQ ID NO:33, and wherein the polypeptide has the asparagines (N) in each of the positions 10 and 45 mutated to a histidine (H), and the asparagine (N) in position 60 mutated to a tyrosine (Y) or a glutamine (Q) relative to SEQ ID NO: 1-4 and 6-9.

Alternatively, according to one embodiment, the kappa light chain-binding domain of Peptostreptococcus Protein L has at least 90%, 95% or 98% sequence identity, or a 77.5% sequence similarity as determined by BLOSUM matrix of 75, with a gap open penalty of 12, a gap extension penalty of 3, with the amino acid sequence SEQ ID NO:34, and wherein the polypeptide has the asparagines (N) in each of the positions 6 and 41 mutated to a histidine (H), and the asparagine (N) in position 56 mutated to a tyrosine (Y) or a glutamine (Q) relative to any one of SEQ ID NO:34.

Thus, according to one embodiment, the kappa light chain-binding polypeptide has an amino acid sequence selected from the group comprising the sequences defined by any one of SEQ ID NO: 35, SEQ ID NO: 36, SEQ ID NO: 37 and SEQ ID NO: 38.

(C2b: N10H, N45H, N60Y mutations)
SEQ ID NO 35
PKEEVTIKVHLIFADGKTQTAEFKGTFEEATAKAYAYADLLAKEHGEYT
ADLEDGGYTIYIKFAGKETPETPEE
(C2b: N10H, N45H, N60Q mutations)
SEQ ID NO 36
PKEEVTIKVHLIFADGKTQTAEFKGTFEEATAKAYAYADLLAKEHGEYT
ADLEDGGYTIQIKFAGKETPETPEE
(C3b: N10H, N45H, N60Y mutations)
SEQ ID NO 37
PKEEVTIKVHLIFADGKIQTAEFKGTFEEATAKAYAYADLLAKEHGEYT
ADLEDGGYTIYIKFAGKETPETPEE
(C3b: N10H, N45H, N60Q mutations)
SEQ ID NO 38
PKEEVTIKVHLIFADGKIQTAEFKGTFEEATAKAYAYADLLAKEHGEYT
ADLEDGGYTIQIKFAGKETPETPEE

Additionally, it was found that the N57Y mutation in the C2b-domain and C3b-domain, in combination with the N60Q mutation, gave rise to a good stability and a good affinity.

According to yet an embodiment, the kappa light chain-binding polypeptide has an amino acid sequence selected from the group comprising the sequences defined by any one of SEQ ID NO: 39, SEQ ID NO:40, SEQ ID NO: 41 and SEQ ID NO:42.

(C4: N10H, N45H, N60Y mutations)
SEQ ID NO: 39
PKEEVTIKVHLIFADGKTQTAEFKGTFEEATAEAYRYADLLAKVHGEYT
ADLEDGGYTIYIKFAGKEQPGENPG
(C4: N10H, N45H, N60Q mutations)
SEQ ID NO: 40
PKEEVTIKVHLIFADGKTQTAEFKGTFEEATAEAYRYADLLAKVHGEYT
ADLEDGGYTIQIKFAGKEQPGENPG
(D1: N10H, N45H, N60Y mutations)
SEQ ID NO: 41
PKEEVTIKAHLIFADGKTQTAEFKGTFEEATAEAYRYADLLAKVHGEYT
ADLEDGGYTIYIKFAGKEQPGEN
(D1: N10H, N45H, N60Q mutations)
SEQ ID NO: 42
PKEEVTIKAHLIFADGKTQTAEFKGTFEEATAEAYRYADLLAKVHGEYT
ADLEDGGYTIQIKFAGKEQPGEN

According to yet an embodiment, based on the truncated versions SEQ ID NO:s 10-18, SEQ ID NO: 32 and SEQ ID NO: 34, the kappa light chain-binding polypeptide has an amino acid sequence selected from the group comprising the sequences defined by any one of SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59 or SEQ ID NO:60.

(B1_trunc, N6H, N41H, N56Y mutations)
SEQ ID NO: 43
VTIKAHLIFANGSTQTAEFKGTFEKATSEAYAYADTLKKDHGEYTVDVA
DKGYTLYIKFAG
(B1_trunc, N6H, N41H, N56Q mutations)
SEQ ID NO: 44
VTIKAHLIFANGSTQTAEFKGTFEKATSEAYAYADTLKKDHGEYTVDVA
DKGYTLQIKFAG
(B2_trunc, N6H, N41H, N56Y mutations)
SEQ ID NO: 45
VTIKAHLIYADGKTQTAEFKGTFEEAAAEAYRYADALKKDHGEYTVDVA
DKGYTLYIKFAG
(B2_trunc, N6H, N41H, N56Q mutations)
SEQ ID NO: 46
VTIKAHLIYADGKTQTAEFKGTFEEAAAEAYRYADALKKDHGEYTVDVA
DKGYTLQIKFAG
(B3_trunc, N6H, N41H, N56Y mutations)
SEQ ID NO: 47
VTIKAHLIYADGKTQTAEFKGTFEEATAEAYRYADLLAKEHGKYTVDVA
DKGYTLYIKFAG
(B3_trunc, N6H, N41H, N56Q mutations)
SEQ ID NO: 48
VTIKAHLIYADGKTQTAEFKGTFEEATAEAYRYADLLAKEHGKYTVDVA
DKGYTLQIKFAG
(B4_trunc, N6H, N41H, N56Y mutations)
SEQ ID NO: 49
VTIKAHLIYADGKTQTAEFKGTFAEATAEAYRYADLLAKEHGKYTADLE
DGGYTIYIRFAG
(B4_trunc, N6H, N41H, N56Q mutations)
SEQ ID NO: 50
VTIKAHLIYADGKTQTAEFKGTFAEATAEAYRYADLLAKEHGKYTADLE
DGGYTIQIRFAG
(B5_trunc, N6H, N41H, N56Y mutations)
SEQ ID NO: 51
VTIKEHIYFEDGTVQTATFKGTFAEATAEAYRYADLLSKEHGKYTADLE
DGGYTIYIRFAG
(B5_trunc, N6H, N41H, N56Q mutations)
SEQ ID NO: 52
VTIKEHIYFEDGTVQTATFKGTFAEATAEAYRYADLLSKEHGKYTADLE
DGGYTIQIRFAG
(C2b_trunc, N6H, N41H, N56Y mutations)
SEQ ID NO: 53
VTIKVHLIFADGKTQTAEFKGTFEEATAKAYAYADLLAKEHGEYTADLE
DGGYTIYIKFAG
(C2b_trunc, N6H, N41H, N56Q mutations)
SEQ ID NO: 54
VTIKVHLIFADGKTQTAEFKGTFEEATAKAYAYADLLAKEHGEYTADLE
DGGYTIQIKFAG
(C3b_trunc, N6H, N41H, N56Y mutations)
SEQ ID NO: 55
VTIKVHLIFADGKIQTAEFKGTFEEATAKAYAYADLLAKEHGEYTADLE
DGGYTIYIKFAG
(C3b_trunc, N6H, N41H, N56Q mutations)
SEQ ID NO: 56
VTIKVHLIFADGKIQTAEFKGTFEEATAKAYAYADLLAKEHGEYTADLE
DGGYTIQIKFAG
(C4_trunc, N6H, N41H, N56Y mutations)
SEQ ID NO: 57
VTIKVHLIFADGKTQTAEFKGTFEEATAEAYRYADLLAKVHGEYTADLE
DGGYTIYIKFAG
(C4_trunc, N6H, N41H, N56Q mutations)
SEQ ID NO: 58
VTIKVHLIFADGKTQTAEFKGTFEEATAEAYRYADLLAKVHGEYTADLE
DGGYTIQIKFAG
(D1_trunc, N6H, N41H, N56Y mutations)
SEQ ID NO: 59
VTIKAHLIFADGKTQTAEFKGTFEEATAEAYRYADLLAKVHGEYTADLE
DGGYTIYIKFAG
(D1_trunc, N6H, N41H, N56Q mutations)
SEQ ID NO: 60
VTIKAHLIFADGKTQTAEFKGTFEEATAEAYRYADLLAKVHGEYTADLE
DGGYTIQIKFAG

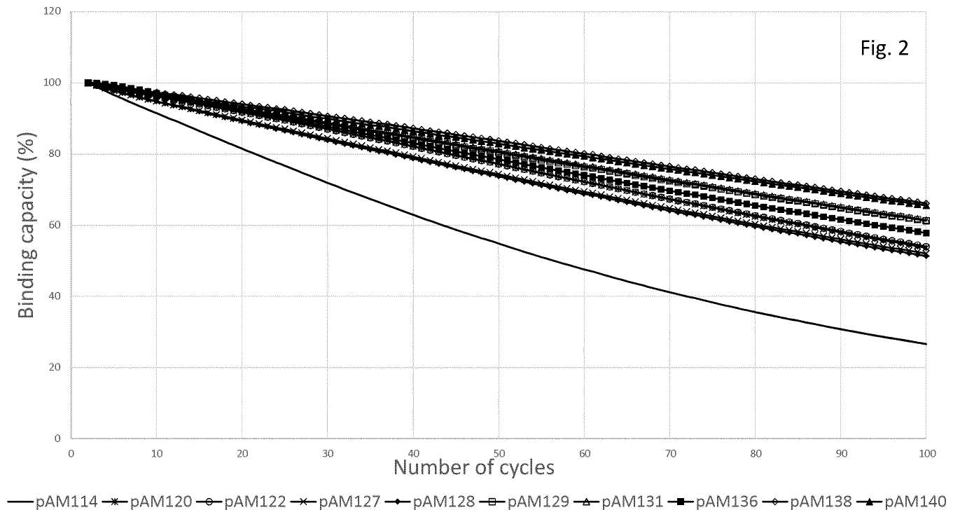

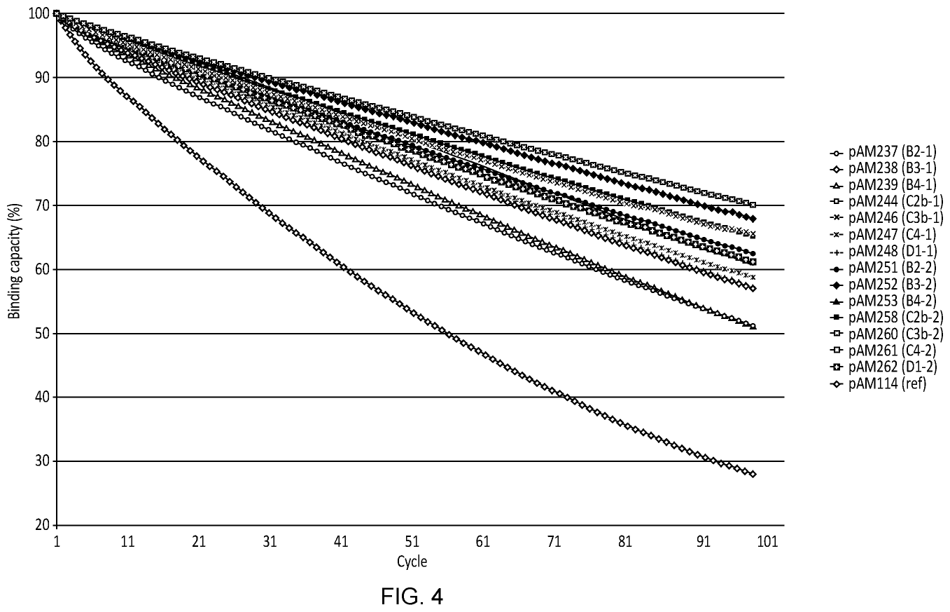

The inventors have thus shown that the mutations N10H, N45H, and N60Y or N60Q, have a positive impact on the alkaline stability of the above-specified Protein L domains. From FIG. 4 it is clearly shown that these mutations greatly improve the alkaline stability compared to wtB3 (pAM114). As shown in FIG. 2, the N60Y/Q mutation also confers an improved alkali stability in comparison with a reference Protein L ligand, herein represented by pAM 128 (SEQ ID NO: 20). As can further be seen in FIG. 4 the alkali stability is domain dependent.

According to yet another embodiment, there is provided a multimer comprising any one of the kappa light chain-binding polypeptides disclosed above. The multimer comprises at least two polypeptides, and may comprise 2, 3, 4, 5, 6, 7, 8 or 9 polypeptides. Thus, the multimer can e.g. be a dimer, a trimer, a tetramer, a pentamer, a hexamer, a heptamer, an octamer or a nonamer. It can be a homomultimer, where all the polypeptides in the multimer are identical or it can be a heteromultimer, where at least one polypeptide differs from the others. Advantageously, all the polypeptides in the multimer are alkali stable, such as by comprising the mutations disclosed above. The polypeptides can be linked to each other directly by peptide bonds between the C-terminal and N-terminal ends of the polypeptides. Alternatively, two or more units in the multimer can be linked by linkers comprising oligomeric or polymeric species, such as elements comprising up to 15 or 30 amino acids, such as 1-5, 1-10 or 5-10 amino acids. Thus, such a multimer may additionally comprise a linker, spacer, or additional amino acid(s) not being involved in the kappa light chain-binding function. Such a linker, spacer or additional amino acid(s) may be positioned between two polypeptides and/or at either of the N-terminal or C-terminal ends of the multimer.

According to yet another embodiment, there is provided a nucleic acid encoding any one of the kappa light chain-binding polypeptides disclosed above, or the multimer disclosed above. Furthermore, according to one embodiment there is provided a vector comprising the above-mentioned nucleic acid. Said vector may comprise further elements such as signal peptides, enhancers, promotors, identification tags, identification or selection markers, and/or purification tags. A non-limiting example of a promoter is the T5 promoter. A non-limiting example of a signal peptide is a OmpA periplasmic signal peptide. Yet another embodiment provides for an expression system, in order to express the polypeptides or multimers disclosed above. The skilled person has the knowledge to choose the particular further elements such as disclosed above, depending on the cloning strategy, expression strategy etc. The expression system may be in a cell culture, or it may be cell-free expression system.

The kappa light chain-binding polypeptides disclosed herein are suitable for use in a separation matrix, coupled to a solid support, for the purpose of separation of any antibodies or antibody fragments comprising a subclass 1, 3 or 4 kappa light chain.

The polypeptides of the present invention are disclosed in more detail in the non-limiting examples below.

### EXAMPLES

### Mutagenesis and Expression of Protein

Monomer constructs were designed according to the following. The Protein L domain starting sequences were SEQ ID NO:s 1-9 and SEQ ID NO: 19.

The Protein L inserts used in the experiments were ordered from IDT, Integrated DNA Technologies (https://eu.idtdna.com). The inserts were cleaved with SalI and BamHI. A pAM113 vector was cut with SalI and BamHI and dephosphorylated. Thereafter the Protein L inserts were ligated into the pAM113 vector. The plasmids were transformed into Top10 chemo-competent E. coli using standard conditions. Standard procedure for transformation was typically, mixing 2 μl 5×KCM (5×KCM=0.5 M KCl, 0.15 M CaCl₂, 0.25 M MgCl₂)+8 μl ligation mix. 10 μl chemo-competent E. coli were added and incubated on ice 20 min followed by incubation at RT for 10 min. 180 μl SOC medium (from Invitrogen, Ref. 15544-034) was added and incubated 1 hr at 37° C. 200 rpm. 200 μl was plated on agar plates supplemented with 100 μg/ml carbenicillin followed by incubation over-night (o/n) at 37° C.

Colonies were inoculated and cultured overnight, followed by sequencing at a MWG Eurofins GATC in order to verify the sequences. Thereafter the Protein L variants were re-transformed into κ12-017 E. coli cells. 200 μl was plated on agar plates supplemented with 100 μg/ml carbenicillin and incubated o/n.

A colony of each protein L variant was inoculated in 4 ml LB (2.5% (w/v) Miller's LB Broth Base from Invitrogen; Ref. 12795027, pH7. LB broth base per liter MilliQ) supplemented with 200 μg/ml carbenicillin in 14 ml falcon tubes and incubated at 37° C. O/N 200 rpm. 500 μl of o/n culture was inoculated in 50 ml Terrific Broth (TB) (Broth: 2 L: 24 g peptone+48 g yeast extract+8 ml glycerol (85%); Potassium phosphate buffer: 11.55 g KH₂PO₄ (MW 136.08)+82.15 g K₂HPO₄*3H₂O (MW 288.23); Broth and potassium phosphate solutions were prepared separately and autoclaved, thereafter 100 ml potassium phosphate buffer was added to 900 ml broth) supplemented with 100 μg/ml carbenicillin in a 250 ml shake flask to obtain a starting OD₆₀₀ nm=0.05. The flasks were incubated at 37° C., 130 rpm for 2 h 45 min until OD₆₀₀ nm=0.7. 25 μl Isopropyl β-d-1-thiogalactopyranoside (IPTG) of stock concentration 1 M was added to each flask to obtain a final IPTG concentration of 0.5 mM and the flasks were incubated o/n at 27° C., 130 rpm.

The cells were centrifuged at 8000×g for 5 min at RT. The supernatant was discarded, and the pellets were resuspended in 10 ml GraviTrap binding buffer (125 ml 8×PBS+10 ml 2M imidazole+865 ml StAq (total vol: 1000 ml)) and transferred to a 15 ml falcon tube. The tubes were heated in a water bath at 80-85° C. for 10 min followed by centrifugation at 8000×g for 15 min. The samples were filtered with 0.2 μM sterile filter and stored at 4° C.

The protein L molecules were purified using GraviTrap from Cytiva according to protocol. Column storage liquid was poured off. Each column was equilibrated with 10 ml binding buffer (125 ml 8×PBS+10 ml 2 M imidazole+865 ml StAq for a total volume of 1000 ml), followed by addition of 10 mL sample. Each column was washed with 2×10 ml binding buffer after loading. Each sample was eluted using 3 ml elution buffer (12.5 ml 8×PBS+25 ml 2M imidazole+62.5 ml StAq for a total volume of 100 ml). Eluate volume of 500 μl was saved from each sample.

Each purified protein L molecule was then buffer exchanged using PD10 desalting columns according to the Cytiva recommended protocol. The column storage liquid was poured off, each column was equilibrated with 5×5 ml PBS, 2.5 ml sample was applied, 500 μl PBS was applied and the flow through was discarded, 3 ml PBS was applied, and the eluted molecules were collected and stored at 4° C. Protein concentration of protein L molecules was measured using Nanodrop with absorbance at 280 nm.

### Experimental Part I

### Example 1—Affinity Test for B3 Domains

The protein L molecules were investigated for apparent affinity on a Biacore 8K+. The analyte used to test the affinity was polyclonal IgG (Gammanorm, Octapharma, Sweden).

The protein L variants were immobilized using amine coupling onto CM5 Chips (Cytiva, Sweden) (see Biacore Sensor Surface Handbook, https://cdn.cytivalifesciences.com/dmm3bwsv3/AssetStream.aspx?mediaformatid=10061&destinationid=10016&assetid=16475).

EDC/NHS activated CM5 surface in 420 s flow rate 10 μl/min over both flow cells, followed by a wash of the system (not sensor surface) with ethanolamine. The variants in immobilization buffer were injected over flow cell 2 and immobilized on activated CM5 surface. Ethanolamine was injected over both flow cells, 420 s flow rate 10 μl/min to deactivate surface.

### Solutions:

| | |
|---|---|
| EDC | 0.4M 1-ethyl-3-(3-dimethylaminopropyl)-carbodiimide |
| | in water |
| NHS | 0.1M N-hydroxysuccinimide in water |
| Ethanolamine | 1M ethanolamine-HCl pH 8.5 |
| Ligand | Typically 10 to 50 μg/mL in immobilization buffer |

| Injection procedure: | Flow rate | Contact time |
|---|---|---|
| 1. EDC/NHS (activate the | 10 μL/min | 7 min |
| surface) | | |
| 2. Ethanolamine (does not | WASH | — |
| pass over the surface) | | |
| 3. Ligand | 10 μL/min | 7 min 4. |
| Ethanolamine (deactivate | 10 μL/min | 7 min |
| excess reactive groups) | | |

Thereafter the protein L variants were assessed for their binding and apparent affinity towards IgG (Gammanorm). The following assay conditions were used:
Analyte: Gammanorm diluted in HBS-EP+ (General purpose running buffer; 0.1 M HEPES, 1.5 M NaCl, 0.03 M EDTA and 0.5% v/v Surfactant P20; from Cytiva)
Analyte concentrations: 0.026, 0.053, 0.106, 0.2125, 0.425, 0.85, 1.7, 3.4 μM
Regeneration: 10 mM glycine pH 1.5
Assay: 10 min association→10 min dissociation→2× regeneration; flow: 10 μl/min

Apparent affinity for B3 domain was estimated using two models: 1:1 kinetic and steady state. The evaluation was performed in Biacore Insight Evaluation Software. K_{D} for steady state affinity and 1:1 kinetics were retrieved from the software. The results of the affinity tests are shown in Table 1.

TABLE 1
steady state affinity and kinetic for 1:1 interaction for
B3-domains with indicated mutations at positions 10, 45 and
60 (pAM114 is wt B3 domain, and pAM128 corresponds to the
previously disclosed benchmarking sequence SEQ ID NO: 20).

| | | | | Steady state | Kinetic |
|---|---|---|---|---|---|
| | Pos | Pos | Pos | affinity | 1:1 |
| Construct | 10 | 45 | 60 | (nM) | (nM) |
| pAM114 | N | N | N | 45 | 12 |
| pAM133 | Y | A | N | 46 | 13 |
| pAM125 | Q | H | N | 50 | 15 |
| pAM132 | Y | N | N | 50 | 15 |
| pAM115 | N | A | N | 51 | 12 |
| pAM116 | N | H | N | 51 | 14 |
| pAM123 | Q | N | N | 51 | 13 |
| pAM124 | Q | A | N | 51 | 12 |
| pAM126 | Q | N | Q | 52 | 16 |
| pAM134 | Y | H | N | 55 | 18 |
| pAM130 | Q | H | Q | 57 | 19 |
| pAM119 | N | A | Q | 58 | 16 |
| pAM117 | N | N | Q | 60 | 18 |
| pAM128 | Q | A | Q | 60 | 17 |
| pAM135 | Y | N | Q | 61 | 17 |
| pAM127 | Q | N | Y | 62 | 15 |
| pAM129 | Q | A | Y | 63 | 14 |
| pAM137 | Y | A | Q | 64 | 18 |
| pAM118 | N | N | Y | 68 | 20 |
| pAM120 | N | A | Y | 69 | 19 |
| pAM139 | Y | H | Q | 71 | 22 |
| pAM138 | Y | A | Y | 73 | 18 |
| pAM121 | N | H | Q | 76 | 22 |
| pAM136 | Y | N | Y | 77 | 19 |
| pAM131 | Q | H | Y | 81 | 21 |
| pAM122 | N | H | Y | 89 | 25 |
| pAM140 | Y | H | Y | 96 | 27 |

The mutations do not affect the overall apparent affinity to any great extent. Mutation in position 60 has a slightly negative effect on affinity. Histidine (H) in position 45 together with Tyrosine (Y) in position 60 is not a good combination which leads to lower apparent affinity and “sticky” interaction. A comparison between pAM114 (NNN) and pAM140 (YHY) shows that YHY produces a more unspecific interaction with a faster off-rate.

Regarding selectivity (data not shown), the mutations in the different variants of B3 didn't have a large impact on the selectivity, although histidine in position 45 slightly weakened binding to kappa 4 and tyrosine in position 60 slightly weakened binding to kappa 3. In general, the selectivity is maintained for all mutated variants.

### Example 2—NaOH Stability Test for B3 Domains

The immobilized protein L ligands were assessed for NaOH stability on a Biacore 8K+. The following assay conditions were used:
Analyte: Gammanorm diluted in HBSEP+ (running buffer)
Analyte concentration: 512 μg/ml (3.4 μM)
Regeneration: 10 mM glycine pH 1.5
NaOH concentration: 100 mM
Assay (one cycle, total 100 cycles): 10 min analyte association→1 min analyte dissociation→10 min NaOH injection→1 min NaOH dissociation→1 min wait→2× regeneration→1 min wait. Flow: 10 μl/min

The assay step was repeated 100 cycles. Cycle 1 was excluded due to a sharp decline in RU (response) between cycle 1 and 2. Therefore cycles 2-100 were used. Results are shown in Table 2 below.

TABLE 2
NaOH Stability for B3-domain (pAM114 is wt B3 domain
and pAM128 corresponds to SEQ ID NO: 20).

| | Pos | Pos | Pos | % Capacity after |
|---|---|---|---|---|
| Construct | 10 | 45 | 60 | 100 cycles |
| pAM138* | Y | A | Y | 66 |
| pAM140* | Y | H | Y | 66 |
| pAM131* | Q | H | Y | 61 |
| pAM129* | Q | A | Y | 61 |
| pAM139 | Y | H | Q | 58 |
| pAM136* | Y | N | Y | 58 |
| pAM130 | Q | H | Q | 56 |
| pAM137 | Y | A | Q | 56 |
| pAM122* | N | H | Y | 54 |
| pAM120* | N | A | Y | 54 |
| pAM127* | Q | N | Y | 52 |
| pAM128* | Q | A | Q | 51 |
| pAM134 | Y | H | N | 49 |
| pAM135 | Y | N | Q | 48 |
| pAM121 | N | H | Q | 47 |
| pAM125 | Q | H | N | 46 |
| pAM119 | N | A | Q | 46 |
| pAM133 | Y | A | N | 45 |
| pAM126 | Q | N | Q | 43 |
| pAM118 | N | N | Y | 42 |
| pAM116 | N | H | N | 41 |
| pAM124 | Q | A | N | 39 |
| pAM115 | N | A | N | 38 |
| pAM132 | Y | N | N | 37 |
| pAM117 | N | N | Q | 33 |
| pAM123 | Q | N | N | 31 |
| pAM114* | N | N | N | 27 |

A selection of the results (marked with *) above are shown in FIG. 2. In contrast to apparent affinity, position 60 is the most important for alkaline stability and Tyrosine (Y) is best amino acid. Alanine (A) and Histidine (H) are equally good and better than Asparagine (N) in position 45. Position 10 is not as crucial as the other positions and Tyrosine (Y) is better than Glutamine (Q). Therefore, the ranking of apparent affinity and NaOH stability are mirror images, i.e. low affinity leads to high alkaline stability and vice versa.

### Example 3—Elution Profiles for B3 Domain

The immobilized protein L ligands were assessed for the possibility of using milder elution conditions. The following assay conditions were used:
Analyte: Gammanorm diluted in HBS-EP+ (General purpose running buffer; 0.1 M HEPES, 1.5 M NaCl, 0.03 M EDTA and 0.5% v/v Surfactant P20; from Cytiva)
Analyte concentrations: 512 μg/ml (3.4 μM)
Regeneration: 50 mM citrate buffer pH 2, 2.5, 3 & 3.5
Assay: 10 min analyte association→1 min analyte dissociation→1 min pH 3.5 injection→1 min pH 3.5 dissociation→1 min pH 3 injection→1 min pH 3 dissociation→1 min pH 2.5 injection→1 min pH 2.5 dissociation→1 min pH 2 injection→1 min pH 2 dissociation; flow: 10 μl/min.

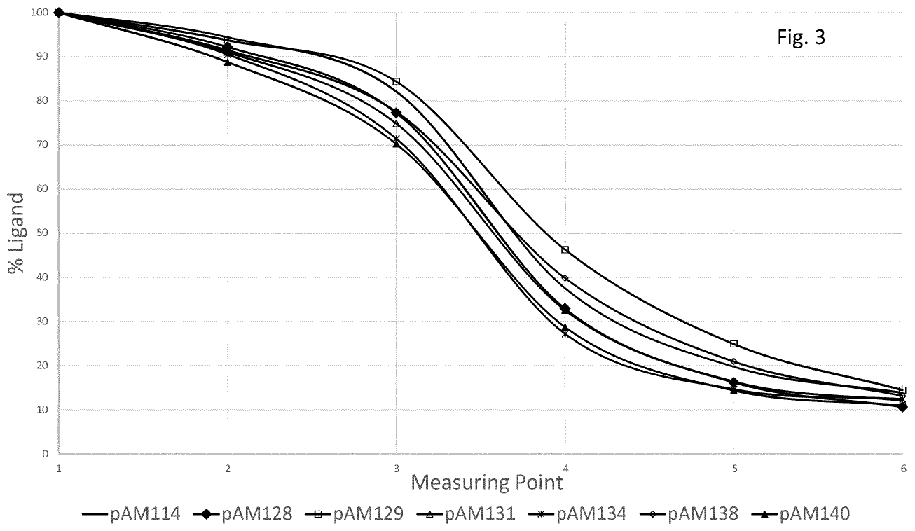

The results for a selection of the variants are shown in FIG. 3. The elution assay revealed no large differences on elution profile between the different B3 variants although histidine in position 45 resulted in faster elution, probably due to a higher off-rate from hydrophobic interaction.

### Example 4—Domains B1, B2, B4, B5, C1-C4 and D1

Biacore results for the remaining domains, being performed as in Examples 1-2, with regards to apparent affinity and NaOH stability are summarized in Tables 3-8.

TABLE 3
Apparent affinity for domains B1, B2, B4, B5.

| | | | | Steady state |
|---|---|---|---|---|
| | Pos | Pos | pos | affinity |
| Construct | 10 | 45 | 60 | KD (nM) |
| B1 | | | | |
| pAM141 | N | N | N | 48 |
| pAM154 | Q | A | Q | 65 |
| pAM167 | Y | H | Y | 97 |
| B2 | | | | |
| pAM143 | N | N | N | 36 |
| pAM169 | Y | H | Y | 76 |
| pAM156 | Q | A | Q | 92 |
| B4 | | | | |
| pAM144 | N | N | N | 38 |
| pAM157 | Q | A | Q | 38 |
| pAM170 | Y | H | Y | 49 |
| B5 | | | | |
| pAM145 | N | N | N | 11 |
| pAM180 | N | H | N | 21 |
| pAM158 | Q | A | Q | 24 |
| pAM171 | Y | H | Y | 40 |

As shown in Table 3, the domains B1, B2, B4 and B5 exhibit similar characteristics as B3 where YHY combination has a negative effect on the affinity. Mutation of additional asparagines in B1, such as in position 15, lowers the affinity (data not shown).

TABLE 4
NaOH stability for domains B1, B2, B4, B5.

| | Pos | Pos | pos | % Binding capacity |
|---|---|---|---|---|
| Construct | 10 | 45 | 60 | after 100 cycles |
| B1 | | | | |
| pAM167 | Y | H | Y | 43 |
| pAM154 | Q | A | Q | 25 |
| pAM141 | N | N | N | 7 |
| B2 | | | | |
| pAM169 | Y | H | Y | 61 |
| pAM156 | Q | A | Q | 47 |
| pAM143 | N | N | N | 17 |
| B4 | | | | |
| pAM170 | Y | H | Y | 57 |
| pAM157 | Q | A | Q | 40 |
| pAM144 | N | N | N | 22 |
| B5 | | | | |
| pAM171 | Y | H | Y | 42 |
| pAM180 | N | H | N | 29 |
| pAM158 | Q | A | Q | 21 |
| pAM145 | N | N | N | 16 |

As is seen in Table 4, YHY combination increases the stability. Mutation of additional asparagines in B1, such as in position 15, increases the stability (data not shown).

TABLE 5
Apparent affinity for domains C1-C4.

| | | | | | | Steady state |
|---|---|---|---|---|---|---|
| | | | | | | affinity |
| Construct | Pos 10 | Pos 39 | Pos 45 | Pos 57 | Pos 60 | KD (nM) |
| C1 | | | | | | |
| pAM146 | N | | N | | N | 61 |
| pAM159 | Q | | A | | Q | 103 |
| pAM172 | Y | | H | | Y | 110 |
| C2 | | | | | | |
| pAM148 | N | | N | | N | 35 |
| pAM162 | Q | | A | Y | Q | 39 |
| pAM149 | N | | N | Y | N | 45 |
| pAM175 | Y | | H | Y | Y | 57 |
| pAM174 | Y | | H | | Y | 105 |
| pAM161 | Q | | A | | Q | 110 |
| C3 | | | | | | |
| pAM151 | N | D | N | Y | N | 30 |
| pAM164 | Q | D | A | Y | Q | 39 |
| pAM150 | N | | N | | N | 40 |
| pAM177 | Y | D | H | Y | Y | 58 |
| pAM176 | Y | | H | | Y | 137 |
| pAM163 | Q | | A | | Q | 188 |
| C4 | | | | | | |
| pAM152 | N | | N | | N | 24 |
| pAM165 | Q | | A | | Q | 38 |
| pAM178 | Y | | H | | Y | 45 |

As seen in Table 5, the C1 and C4 domains exhibit similar characteristics as the B3-domain where YHY combination has a negative effect on affinity. As can also be seen in Table 5, the additional mutation of the asparagine in position 39 in C3, and of the asparagine in position 57 in both C2 and C3, increases the affinity.

TABLE 6
NaOH stability for domains C1-C4.

| | | | | | | % Binding |
|---|---|---|---|---|---|---|
| | | | | | | capacity |
| | | | | | | after |
| Construct | Pos 10 | Pos 39 | Pos 45 | Pos 57 | Pos 60 | 100 cycles |
| C1 | | | | | | |
| pAM172 | Y | | H | | Y | 41 |
| pAM159 | Q | | A | | Q | 28 |
| pAM146 | N | | N | | N | 4 |
| C2 | | | | | | |
| pAM175 | Y | | H | Y | Y | 61 |
| pAM162 | Q | | A | Y | Q | 54 |
| pAM174 | Y | | H | | Y | 36 |
| pAM149 | N | | N | Y | N | 32 |
| pAM161 | Q | | A | | Q | 24 |
| pAM148 | N | | N | | N | 10 |
| C3 | | | | | | |
| pAM177 | Y | D | H | Y | Y | 67 |
| pAM164 | Q | D | A | Y | Q | 62 |
| pAM176 | Y | | H | | Y | 42 |
| pAM151 | N | D | N | Y | N | 38 |
| pAM163 | Q | | A | | Q | 22 |
| pAM150 | N | | N | | N | 9 |
| C4 | | | | | | |
| pAM178 | Y | | H | | Y | 56 |
| pAM165 | Q | | A | | Q | 56 |
| pAM152 | N | | N | | N | 33 |

As seen in Table 6, C1 and C4 domains exhibit similar characteristics as B3 where YHY combination increases stability. However, in the C2 and C3 domains, tyrosine (Y) in pos. 57 has a greater impact on NaOH stability than Y in pos. 60. Tyrosine in pos. 57 in combination with Q60 has a good affinity and stability (see pAM162 and 164). Mutation of additional asparagines in domains C1 and C3, such as position 20, can improve the affinity and increase stability (data not shown).

TABLE 7
Apparent affinity for domain D1.

| D1 | | | | Steady state |
|---|---|---|---|---|
| Construct | Pos 10 | Pos 45 | Pos 60 | affinity KD (nM) |
| pAM153 | N | N | N | 32 |
| pAM166 | Q | A | Q | 41 |
| pAM179 | Y | H | Y | 75 |

TABLE 8
NaOH stability for domain D1.

| D1 | | | | % Binding capacity |
|---|---|---|---|---|
| Construct | Pos 10 | Pos 45 | pos 60 | after 100 cycles |
| pAM179 | Y | H | Y | 54 |
| pAM166 | Q | A | Q | 47 |
| pAM153 | N | N | N | 23 |

As seen in Tables 7 and 8, the D1-domain exhibits similar characteristics as B3.

### Summary Experimental Part I

These experiments above show that for most domains Y in position 60 is most crucial for NaOH stability. For the domains C2 and C3, Y in position 57 is also important for NaOH stability.

The results from affinity measurements indicate that affinity has a negative correlation with NaOH stability and “stickiness”, i.e. high affinity correlates to lower stability and lower “stickiness”. The exceptions here are pAM162 (C2) and pAM164 (C3) both of which have good affinity and stability.

When the domains are compared to each other with regards to affinity and NaOH stability, the domains B3, C2 and C3 can be considered “good” domains whereas B1 and C1 can be considered “less good” domains. For the domains C2 and C3, additional mutations in position 57 provide improvement regarding both the stability and the affinity.

The two individual variants pAM162 and pAM164 can be regarded as the best “allrounders” i.e. showing good affinity and good stability.

Based on the results of Experimental Part I, the inventors found it interesting to further investigate the most promising mutations on all the Protein L domains.

### Experimental Part II

### Example 5—Construction of Expression Vectors N10H, N45H, N60Q/N60H for all Protein L Domains

In view of the above, and previous work within the field of Protein L ligands, it was decided to proceed to look at the impact of the specific mutations N10H, N45H and N60Y/N60Q on all the domains tested above. The protein L-variants in Table 9 were ordered from IDT as disclosed above and expressed as disclosed above.

In this set, a C2b domain and a C3b domain are included, in view of the results in Tables 5 and 6 above. The C2b domain comprises an additional N57Y mutation. The C3b domain comprises an additional N39D and N57Y mutations.

TABLE 9
Constructed Protein L-variants

| | | Pos | Pos | Pos |
|---|---|---|---|---|
| Construct | Name | 10 | 45 | 60 |
| pAM235 | pL68 B1-1 | H | H | Q |
| pAM236 | pL69 B1b-1 | H | H | Q |
| pAM237 | pL70 B2-1 | H | H | Q |
| pAM238 | pL71 B3-1 | H | H | Q |
| pAM239 | pL72 B4-1 | H | H | Q |
| pAM240 | pL73 B5b-1 | H | H | Q |
| pAM241 | pL74 C1-1 | H | H | Q |
| pAM242 | pL75 C1b-1 | H | H | Q |
| pAM243 | pL76 C2-1 | H | H | Q |
| pAM244 | pL77 C2b-1 | H | H | Q |
| pAM245 | pL78 C3-1 | H | H | Q |
| pAM246 | pL79 C3b-1 | H | H | Q |
| pAM247 | pL80 C4-1 | H | H | Q |
| pAM248 | pL81 D1-1 | H | H | Q |
| pAM249 | pL82 B1-2 | H | H | Y |
| pAM250 | pL83 B1b-2 | H | H | Y |
| pAM251 | pL84 B2-2 | H | H | Y |
| pAM252 | pL85 B3-2 | H | H | Y |
| pAM253 | pL86 B4-2 | H | H | Y |
| pAM254 | pL87 B5b-2 | H | H | Y |
| pAM255 | pL88 C1-2 | H | H | Y |
| pAM256 | pL89 C1b-2 | H | H | Y |
| pAM257 | pL90 C2-2 | H | H | Y |
| pAM258 | pL91 C2b-2 | H | H | Y |
| pAM259 | pL92 C3-2 | H | H | Y |
| pAM260 | pL93 C3b-2 | H | H | Y |
| pAM261 | pL94 C4-2 | H | H | Y |
| pAM262 | pL95 D1-2 | H | H | Y |

### Example 6—Apparent Affinity Measurement Against Analyte Gammanorm

The immobilised protein L ligands were assessed for their binding and apparent affinity towards IgG (Gammanorm). The following assay conditions were used:
Analyte: Gammanorm diluted in HBS-EP+ (General purpose running buffer; 0.1 M HEPES, 1.5 M NaCl, 0.03 M EDTA and 0.5% v/v Surfactant P20; from Cytiva)
Analyte concentrations: 0.026, 0.053, 0.106, 0.225, 0.425, 0.85, 1.7, 3.4 μM
Regeneration: 10 mM glycine pH 1.5
Assay: 10 min association→10 min dissociation→2× regeneration. Flow: 10 μl/min

The evaluation was performed in Biacore Insight Evaluation Software, where kinetic and affinity fitting to sensorgrams (not shown) were performed. K_{D} for steady state affinity and 1:1 kinetics were retrieved from the software. The interactions were evaluated to be “sticky” or “good”, depending on the curvature of the sensorgrams.

TABLE 10
Protein L variants with specified amino acids at position 10, 45 and 60, with the KD values
for steady state interaction and 1:1 kinetics and the quality of the interaction to IgG.
The table is sorted on the lowest steady state affinity KD first. pAM114 is wt B3 domain.

| | | | | | Steady state affinity | Kinetic 1:1 | |
|---|---|---|---|---|---|---|---|
| Construct | Domain | Pos 10 | Pos 45 | Pos 60 | KD (nM) | (nM) | Quality |
| pAM247* | C4-1 | H | H | Q | 38 | 9 | Good |
| pAM254* | B5b-2 | H | H | Y | 41 | 7 | Good |
| pAM261* | C4-2 | H | H | Y | 43 | 12 | Good |
| pAM258* | C2b-2 | H | H | Y | 45 | 17 | Good |
| pAM114* | B3 | N | N | N | 46 | 11 | Good |
| pAM262* | D1-2 | H | H | Y | 48 | 11 | Good |
| pAM246* | C3b-1 | H | H | Q | 49 | 17 | Good |
| pAM244* | C2b-1 | H | H | Q | 50 | 18 | Good |
| pAM260* | C3b-2 | H | H | Y | 51 | 18 | Good |
| pAM253* | B4-2 | H | H | Y | 54 | 14 | Good |
| pAM251* | B2-2 | H | H | Y | 55 | 20 | Good |
| pAM248* | D1-1 | H | H | Q | 56 | 16 | Good |
| pAM239* | B4-1 | H | H | Q | 60 | 17 | Good |
| pAM252 | B3-2 | H | H | Y | 69 | 16 | Good |
| pAM237* | B2-1 | H | H | Q | 72 | 27 | Good |
| pAM240* | B5b-1 | H | H | Q | 79 | 15 | Good |
| pAM249 | B1-2 | H | H | Y | 79 | 29 | Sticky |
| pAM238 | B3-1 | H | H | Q | 82 | 25 | Good |
| pAM243 | C2-1 | H | H | Q | 83 | 39 | Sticky |
| pAM257 | C2-2 | H | H | Y | 98 | 47 | Sticky |
| pAM250 | B1b-2 | H | H | Y | 99 | 40 | Sticky |
| pAM235 | B1-1 | H | H | Q | 101 | 42 | Sticky |
| pAM242 | C1b-1 | H | H | Q | 116 | 49 | Sticky |
| pAM259 | C3-2 | H | H | Y | 120 | 55 | Sticky |
| pAM236 | B1b-1 | H | H | Q | 130 | 61 | Sticky |
| pAM245 | C3-1 | H | H | Q | 134 | 65 | Sticky |
| pAM256 | C1b-2 | H | H | Y | 134 | 59 | Sticky |
| pAM241* | C1-1 | H | H | Q | 187 | 87 | Sticky |
| pAM255* | C1-2 | H | H | Y | 324 | 146 | Sticky |

The results show that Protein L domains B1, C1, C2 and C3 have sticky interactions, and there is a correlation between sticky interaction and low affinity. The affinity seems to be domain dependent, and there is no major difference noticed between the HHQ-versions and the HHY versions of the domains.

### Example 7—NaOH Stability Assessment

The immobilised protein L ligands were assessed for NaOH stability. The following assay conditions were used:
Analyte: Gammanorm diluted in HBS-EP+ (General purpose running buffer; 0.1 M HEPES, 1.5 M NaCl, 0.03 M EDTA and 0.5% v/v Surfactant P20; from Cytiva™)
Analyte concentration: 3.4 μM
Regeneration: 10 mM glycine pH 1.5
NaOH concentration: 100 mM
Assay: 10 min analyte association→1 min analyte dissociation→10 min NaOH injection→1 min NaOH dissociation→1 min wait→2× regeneration→1 min wait→repeat 100 cycles. Flow: 10 μl/min

The evaluation was performed in Biacore Insight Evaluation software, where the responses at the report point “Binding late” were collected. Cycle 1 (start-up) and cycle 2 (first cycle of analysis) were excluded from evaluation. The responses in the first evaluation cycle (cycle 3) were set to 100% and the responses in the following 99 cycles were set to a percentage of the responses from the first cycle.

To simplify the comparison of NaOH stability between the variants, the NaOH stability are visualized as a line diagram of decreasing stability over increasing number of cycles, see FIG. 4. In Table 11, the final percentage capacity after 100 cycles for each variant are listed and sorted on highest capacity first.

TABLE 11
Protein L variants with specified amino acids with
the percent binding capacity after 100 cycles of
0.1M NaOH. The table is sorted on the highest capacity
after 100 cycles first. pAM114 is wt B3 domain.

| | | Pos | Pos | Pos | % Capacity after |
|---|---|---|---|---|---|
| Construct | Domain | 10 | 45 | 60 | 100 cycles |
| pAM260* | C3b-2 | H | H | Y | 70 |
| pAM252* | B3-2 | H | H | Y | 68 |
| pAM246* | C3b-1 | H | H | Q | 66 |
| pAM258* | C2b-2 | H | H | Y | 63 |
| pAM251* | B2-2 | H | H | Y | 62 |
| pAM244* | C2b-1 | H | H | Q | 61 |
| pAM253* | B4-2 | H | H | Y | 61 |
| pAM261* | C4-2 | H | H | Y | 61 |
| pAM262* | D1-2 | H | H | Y | 61 |
| pAM247* | C4-1 | H | H | Q | 59 |
| pAM238* | B3-1 | H | H | Q | 57 |
| pAM248* | D1-1 | H | H | Q | 57 |
| pAM237 | B2-1 | H | H | Q | 51 |
| pAM239 | B4-1 | H | H | Q | 51 |
| pAM256 | C1b-2 | H | H | Y | 51 |
| pAM250 | B1b-2 | H | H | Y | 46 |
| pAM249 | B1-2 | H | H | Y | 45 |
| pAM254 | B5b-2 | H | H | Y | 44 |
| pAM242 | C1b-1 | H | H | Q | 43 |
| pAM255 | C1-2 | H | H | Y | 41 |
| pAM257 | C2-2 | H | H | Y | 41 |
| pAM259 | C3-2 | H | H | Y | 41 |
| pAM236 | B1b-1 | H | H | Q | 35 |
| pAM243* | C2-1 | H | H | Q | 35 |
| pAM245* | C3-1 | H | H | Q | 35 |
| pAM241* | C1-1 | H | H | Q | 34 |
| pAM235 | B1-1 | H | H | Q | 32 |
| pAM114 | B3 | N | N | N | 29 |
| pAM240* | B5b-1 | H | H | Q | 22 |

The results show that all variants apart from pAM240 have a better NaOH stability compared to pAM114 (wt B2 domain). The HHY variant seems to have a slightly better NaOH stability compared to HHQ. However, in general both variants are shown to perform well. The NaOH stability is mainly domain dependent, where B5, C1, C2 and C3 show the lowest alkali stability. C3b, B3, C2b, B2, B4, C4 and D1 all show a very good alkali stability, for both of the variants HHY and HHQ.

### Example 8—Assessment of Elution in pH 2-3.5

The immobilised protein L ligands were assessed for the possibility of using milder elution conditions. The following assay conditions were used:
Analyte: Gammanorm diluted in HBS-EP+ (General purpose running buffer; 0.1 M HEPES, 1.5 M NaCl, 0.03 M EDTA and 0.5% v/v Surfactant P20; from Cytiva)
Analyte concentrations: 3.4 μM
Regeneration: 50 mM citrate buffer pH 2, 2.5, 3 & 3.5
Assay: 10 min analyte association→1 min analyte dissociation→1 min pH 3.5 injection→1 min pH 3.5 dissociation→1 min pH 3 injection→1 min pH 3 dissociation→1 min pH 2.5 injection→1 min pH 2.5 dissociation→1 min pH 2 injection→1 min pH 2 dissociation→2× regeneration. Flow: 10 μl/min

The evaluation of the results was performed in Biacore Insight Evaluation Software and compiled in excel. The responses in the report point “Stability late” were collected for 6 different measure points; 1: after analyte injection, 2: pH=3.5, 3: pH=3.0, 4: pH=2.5, 5: pH=2.0 and 6: regeneration at pH=1.5. The responses after analyte injection were set to 100% and the following responses at the following measure points were set as a percentage.

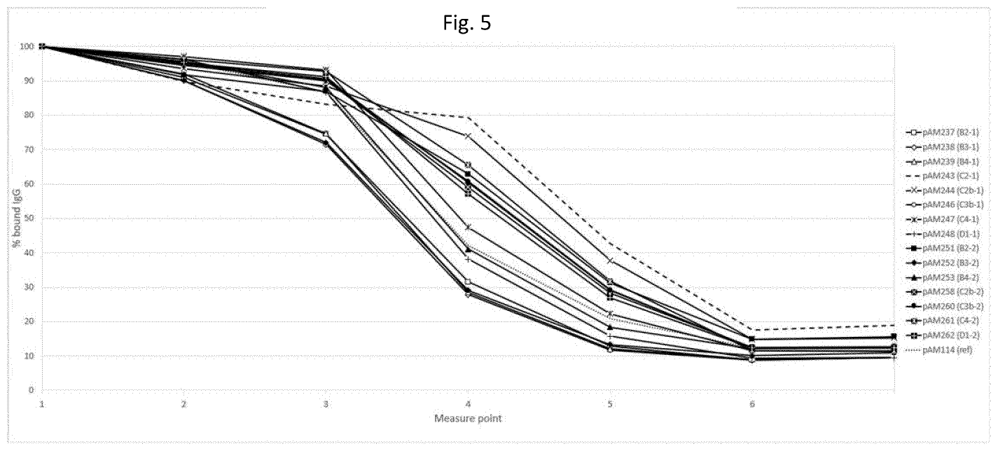

The results are shown in FIG. 5 both the HHY-variant and the HHQ variant of the B2 domain, the B3 domain, the B4 domain, the C2b domain, the C3b domain, the C4 domain and the D1 domain elute easier than the reference C2, which corresponds to a “sticky” interaction. The wtB3 (pAM114) is represented with a dotted line. HHQ variants seems to be slightly easier to elute compared to HHY variants.

### Summary and Conclusion

Below, in Tables 12-14, the results of the above-disclosed experiments relating to affinity and alkali stability are summarized per domain and compared with the wtB3 domain.

From the above disclosed experiments and the summary thereof below, it is clear that the mutations N10H, N45H, and N60Y or N60Q have a positive effect on the NaOH stability on Protein L, and that the affinity is maintained. Additionally, the selectivity to the kappa light chains κ1, κ3 and κ4 is maintained (data not shown). It is notable that the effect is shown on a broader range of Protein L domains. The effect is shown, in particular, for the B2 domain, the B3 domain, the B4 domain, the C2b domain, the C3b domain, the C4 domain and the D1 domain.

TABLE 12
Summary of domains B1-B5. Affinity in the left part, NaOH stability in the right part.

| | | | | | Steady | | | | | | | | |
|---|---|---|---|---|---|---|---|---|---|---|---|---|---|
| | | | | | state | | | | | | | | % capacity |
| | | Pos | Pos | Pos | affinity KD | Kinetic 1:1 | | | | | | | after |
| Construct | Domain | 10 | 45 | 60 | (nM) | (nM) | Quality | Construct | Domain | Pos 10 | Pos 45 | Pos 60 | 100 cycles |
| pAM114 | B3 | N | N | N | 46 | 11 | Good | pAM250 | B1b-2 | H | H | Y | 46 |
| pAM249 | B1-2 | H | H | Y | 79 | 29 | Sticky | pAM249 | B1-2 | H | H | Y | 45 |
| pAM250 | B1b-2 | H | H | Y | 99 | 40 | Sticky | pAM236 | B1b-1 | H | H | Q | 35 |
| pAM235 | B1-1 | H | H | Q | 101 | 42 | Sticky | pAM235 | B1-1 | H | H | Q | 32 |
| pAM236 | B1b-1 | H | H | Q | 109 | 44 | Sticky | pAM114 | B3 | N | N | N | 30 |
| pAM114 | B3 | N | N | N | 46 | 11 | Good | pAM251 | B2-2 | H | H | Y | 62 |
| pAM251 | B2-2 | H | H | Y | 55 | 20 | Good | pAM237 | B2-1 | H | H | Q | 51 |
| pAM237 | B2-1 | H | H | Q | 72 | 27 | Good | pAM114 | B3 | N | N | N | 30 |
| pAM114 | B3 | N | N | N | 46 | 11 | Good | pAM252 | B3-2 | H | H | Y | 68 |
| pAM252 | B3-2 | H | H | Y | 69 | 16 | Good | pAM238 | B3-1 | H | H | Q | 57 |
| pAM238 | B3-1 | H | H | Q | 82 | 25 | Good | pAM114 | B3 | N | N | N | 30 |
| pAM114 | B3 | N | N | N | 46 | 11 | Good | pAM253 | B4-2 | H | H | Y | 61 |
| pAM253 | B4-2 | H | H | Y | 54 | 14 | Good | pAM239 | B4-1 | H | H | Q | 51 |
| pAM239 | B4-1 | H | H | Q | 60 | 17 | Good | pAM114 | B3 | N | N | N | 30 |
| pAM254 | B5b-2 | H | H | Y | 41 | 7 | Good | pAM254 | B5b-2 | H | H | Y | 44 |
| pAM114 | B3 | N | N | N | 46 | 11 | Good | pAM114 | B3 | N | N | N | 30 |
| pAM240 | B5b-1 | H | H | Q | 79 | 15 | Good | pAM240 | B5b-1 | H | H | Q | 22 |

TABLE 13
Summary of the domains C1-C4. Affinity in the left part, NaOH stability in the right part

| | | | | | Steady state | | | | | | | | |
|---|---|---|---|---|---|---|---|---|---|---|---|---|---|
| | | Pos | Pos | Pos | affinity KD | Kinetic 1:1 | | | | | | | % capacity after |
| Construct | Domain | 10 | 45 | 60 | (nM) | (nM) | Quality | Construct | Domain | Pos 10 | Pos 45 | Pos 60 | 100 cycles |
| pAM114 | B3 | N | N | N | 46 | 11 | Good | pAM256 | C1b-2 | H | H | Y | 51 |
| pAM242 | C1b-1 | H | H | Q | 116 | 49 | Sticky | pAM242 | C1b-1 | H | H | Q | 43 |
| pAM256 | C1b-2 | H | H | Y | 134 | 59 | Sticky | pAM255 | C1-2 | H | H | Y | 41 |
| pAM241 | C1-1 | H | H | Q | 187 | 87 | Sticky | pAM241 | C1-1 | H | H | Q | 34 |
| pAM255 | C1-2 | H | H | Y | 324 | 146 | Sticky | pAM114 | B3 | N | N | N | 30 |
| pAM258 | C2b-2 | H | H | Y | 45 | 17 | Good | pAM258 | C2b-2 | H | H | Y | 65 |
| pAM114 | B3 | N | N | N | 46 | 11 | Good | pAM244 | C2b-1 | H | H | Q | 61 |
| pAM244 | C2b-1 | H | H | Q | 50 | 18 | Good | pAM257 | C2-2 | H | H | Y | 41 |
| pAM243 | C2-1 | H | H | Q | 83 | 39 | Sticky | pAM243 | C2-1 | H | H | Q | 35 |
| pAM257 | C2-2 | H | H | Y | 98 | 47 | Sticky | pAM114 | B3 | N | N | N | 30 |
| pAM114 | B3 | N | N | N | 46 | 11 | Good | pAM260 | C3b-2 | H | H | Y | 70 |
| pAM246 | C3b-1 | H | H | Q | 47 | 15 | Good | pAM246 | C3b-1 | H | H | Q | 66 |
| pAM260 | C3b-2 | H | H | Y | 51 | 18 | Good | pAM259 | C3-2 | H | H | Y | 43 |
| pAM245 | C3-1 | H | H | Q | 118 | 54 | Sticky | pAM245 | C3-1 | H | H | Q | 35 |
| pAM259 | C3-2 | H | H | Y | 120 | 55 | Sticky | pAM114 | B3 | N | N | N | 30 |
| pAM247 | C4-1 | H | H | Q | 38 | 9 | Good | pAM261 | C4-2 | H | H | Y | 61 |
| pAM261 | C4-2 | H | H | Y | 43 | 12 | Good | pAM247 | C4-1 | H | H | Q | 59 |
| pAM114 | B3 | N | N | N | 46 | 11 | Good | pAM114 | B3 | N | N | N | 30 |

TABLE 14
Summary of D1 domain. A) affinity, B) NaOH stability
A)

| | | | | | Steady state | Kinetic 1:1 | |
|---|---|---|---|---|---|---|---|
| Construct | Domain | Pos 10 | Pos 45 | Pos 60 | affinity KD (nM) | (nM) | Quality |
| pAM114 | B3 | N | N | N | 46 | 11 | Good |
| pAM262 | D1-2 | H | H | Y | 48 | 11 | Good |
| pAM248 | D1-1 | H | H | Q | 56 | 16 | Good |

B)

| Construct | Domain | Pos 10 | Pos 45 | Pos 60 | % Capacity after 100 cycles |
|---|---|---|---|---|---|
| pAM262 | D1-2 | H | H | Y | 61 |
| pAM248 | D1-1 | H | H | Q | 57 |
| pAM114 | B3 | N | N | N | 30 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties, as if they were individually incorporated.

1. A kappa light chain-binding polypeptide consisting of, consisting essentially of, or comprising at least one mutated binding domain of Peptostreptococcus Protein L,
which domain has at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:10, SEQ ID NO:11, SEQ ID NO:12, SEQ ID NO:13, SEQ ID NO:14, SEQ ID NO:15, SEQ ID NO:16, SEQ ID NO:17 or SEQ ID NO:18,
and wherein the polypeptide has the asparagines in each of the positions 6 and 41 mutated to a histidine, and the asparagine in position 56 mutated to a tyrosine or a glutamine relative to any one of SEQ ID NO:s 10-18.

2. The kappa light chain-binding polypeptide according to claim 1, wherein the binding domain of Peptostreptococcus Protein L has at least 90%, 95% or 98% sequence identity, or a 77.5% sequence similarity as determined by BLOSUM matrix of 75, with a gap open penalty of 12, a gap extension penalty of 3, with any one of the amino acid sequences SEQ ID NO:1, SEQ ID NO:2, SEQ ID NO:3, SEQ ID NO: 4, SEQ ID NO: 5, SEQ ID NO:6, SEQ ID NO:7 or SEQ ID NO:8, and wherein the polypeptide has the asparagines in each of the positions 10 and 45 mutated to a histidine, and the asparagine in position 60 mutated to a tyrosine or a glutamine relative to SEQ ID NO: 1-4 and 6-9; or
wherein the binding domain of Peptostreptococcus Protein L has at least 90%, 95% or 98% sequence identity, or a 77.5% sequence similarity as determined by BLOSUM matrix of 75, with a gap open penalty of 12, a gap extension penalty of 3, with SEQ ID NO: 9 and wherein the polypeptide has the asparagines in each of the positions 9 and 44 mutated to a histidine, and the asparagine in position 59 mutated to a tyrosine or a glutamine relative to SEQ ID NO: 5.

3. The kappa light chain-binding polypeptide according to claim 1, wherein the binding domain of Peptostreptococcus Protein L is selected from the group comprising of a B2 domain, a B3 domain, a B4 domain, a C2 domain, a C3 domain, a C4 domain and a D1 domain.

4. The kappa light chain-binding polypeptide according to claim 3, wherein the binding domain of Peptostreptococcus Protein L is selected from the group comprising of the B3 domain, the C2 domain, the C3 domain and the D-domain.

5. The kappa light chain-binding polypeptide according to claim 1, wherein the domain has a 90%, 95% or 98% sequence identity, or a 77.5% sequence similarity as determined by BLOSUM matrix of 75, with a gap open penalty of 12, a gap extension penalty of 3, with any one of the sequences SEQ ID NO:3, SEQ ID NO:6 or SEQ ID NO:7.

6. The kappa light chain-binding polypeptide according to claim 3, wherein the C2 domain is a domain wherein, additionally, the asparagine in position 57 has been mutated to a tyrosine or a glutamine, such as a tyrosine (Y).

7. The kappa light chain-binding polypeptide according to claim 1, wherein the domain has a 90%, 95% or 98% sequence identity, or a 77.5% sequence similarity as determined by BLOSUM matrix of 75, with a gap open penalty of 12, a gap extension penalty of 3, with SEQ ID NO:31 or SEQ ID NO: 32.

8. The kappa light chain-binding polypeptide according to claim 3, wherein the C3 domain is a domain wherein, additionally, the asparagine in position 57 has been mutated to a tyrosine or a glutamine, such as a tyrosine, and an asparagine in position 39 has been mutated to an aspartic acid.

9. The kappa light chain-binding polypeptide according to claim 1, wherein the domain has a 90%, 95% or 98% sequence identity, or a 77.5% sequence similarity as determined by BLOSUM matrix of 75, with a gap open penalty of 12, a gap extension penalty of 3, with SEQ ID NO:33 or SEQ ID NO: 34.

10. The kappa light chain-binding polypeptide according to claim 1, wherein the polypeptide has a 90%, 95% or 98% sequence identity, or a 77.5% sequence similarity as determined by BLOSUM matrix of 75, with a gap open penalty of 12, a gap extension penalty of 3, with any one of the sequences SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:41, SEQ ID NO:52, SEQ ID NO:53, SEQ ID NO:54, SEQ ID NO:55, SEQ ID NO:56, SEQ ID NO:57, SEQ ID NO:58, SEQ ID NO:59 or SEQ ID NO:60.

11. The kappa light chain-binding polypeptide according to claim 10, wherein the polypeptide has a 90%, 95% or 98% sequence identity, or a 77.5% sequence similarity as determined by BLOSUM matrix of 75, with a gap open penalty of 12, a gap extension penalty of 3, with any one of the sequences SEQ ID NO:21, SEQ ID NO:22, SEQ ID NO:35, SEQ ID NO:36, SEQ ID NO:37, SEQ ID NO:38, SEQ ID NO:47, SEQ ID NO:48, SEQ ID NO:53, SEQ ID NO:54, SEQ ID NO:55 or SEQ ID NO:56.

12. The kappa light chain-binding polypeptide according to claim 10, wherein the polypeptide has a 90%, 95% or 98% sequence identity, or a 77.5% sequence similarity as determined by BLOSUM matrix of 75, with a gap open penalty of 12, a gap extension penalty of 3, with any one of the sequences SEQ ID NO:21, SEQ ID NO:22, SEQ ID NO:47 or SEQ ID NO:48.

13. The kappa light chain-binding polypeptide according to claim 1, further comprising a spacer or a linker N-terminally or C-terminally of the specified amino acid sequence, and/or additional amino acid(s) N-terminally or C-terminally of the specified amino acid sequence.

14. The kappa light chain-binding polypeptide according to claim 1, further comprising at the N-terminus a plurality of amino acid residues originating from the cloning process or constituting a residue from a cleaved off signaling sequence, wherein the number of additional amino acid residues is 15 or less, such as 10 or less or 5 or less.

15. The kappa light chain-binding polypeptide according to claim 1, wherein the kappa light chain-binding polypeptide binds to κ1, κ3 and κ4.

16. A multimer comprising at least two of the polypeptides according to claim 1, such as two, three, four, five, six, seven, eight or nine polypeptides.

17. The multimer according to claim 16, further comprising a linker, spacer, or additional amino acid(s).

18. A nucleic acid encoding the polypeptide according to claim 1.

19. A vector comprising the nucleic acid according to claim 18, optionally further comprising one or more of a signal peptide, enhancer, promotor, identification tag, identification marker, selection marker, and/or purification tag.

20. An expression system comprising the nucleic acid according to claim 18.

21. A separation matrix comprising at least one polypeptide according to claim 1, coupled to a solid support.
